(19) United States
(12) Patent Application Publication
(10) Pub. No.: US 2021/0319441 A1
(43) Pub. Date: Oct. 14, 2021

(54) UTILITY TOKEN-BASED SYSTEMS AND METHODS

(71) Applicant: FOOGAL, INC., Lincoln, NE (US)

(72) Inventor: Todd KNOBEL, Lincoln, NE (US)

(21) Appl. No.: 15/734,546

(22) PCT Filed: May 31, 2019
(86) PCT No.: PCT/US2019/035024

§ 371 Date: Dec. 2, 2020

Related U.S. Application Data
(60) Provisional application No. 62/680,526, filed on Jun. 4, 2018.

(51) Int. Cl. G06Q 20/38; G06Q 20/36; G06Q 20/40; H04L 9/08; H04L 9/32

(52) U.S. Cl. CPC G06Q 20/38215; G06Q 20/36; G06Q 20/3829; G06Q 20/401; G06Q 2220/00; H04L 9/0825; H04L 9/3236; H04L 2209/38; G06Q 20/381

(57) ABSTRACT

Provided herein are systems, methods, and networks for tracking user compliance with a health program using a peer-to-peer distributed ledger computer network. Further provided are methods for stabilizing the price of tokens supplied to the distributed ledger computer network using a central authority or off-chain oracle. In some embodiments, the methods of stabilizing the price of tokens is leveraged in the systems and networks disclosed herein.

### CROSS-REFERENCE

This application claims the benefit of U.S. Provisional Application No. 62/680,526, filed Jun. 4, 2018, which is hereby incorporated by reference in its entirety herein.

### BACKGROUND

According to several leading public health experts, 75% percent of chronic metabolic syndrome diseases could be prevented if individuals would comply with proscribed nutrition guidelines. Failure of compliance with nutrition guidelines contributes to an annual United States (U.S.) healthcare cost of approximately $1.6 trillion, which amounts to roughly $4,950 per person. By comparison, Americans annually spend about $588 billion on groceries, or about $1,820 per person. Thus, the U.S. healthcare system and its constituents will incur lower healthcare costs, if individuals were incentivized to eat healthy and nutritious food.

### SUMMARY

One method of tracking and incentivizing human behavior is through a token economy, or cryptocurrency. Cryptocurrencies enable a secure distributed computing system through decentralization and secure processing of transactions using blockchain. A blockchain consists of a continuous, sequential set of data blocks, each block linked to its successor through cryptography. A block typically contains a cryptographic code or hash of the previous block, a timestamp and transaction data often in a ledger format. Once recorded, the data in any given block cannot be altered retroactively without the alteration of all subsequent blocks, which requires collusion of the network majority.

Decentralization of a distributed ledger blockchain is typically achieved by a peer-to-peer network collectively adhering to a protocol for inter-node communication and validating new blocks. Validation of new blocks is usually carried out by solving complex algorithms, known as proof of work, or from the vested interest a party has in the blockchain determined by the stake and duration of the holding (i.e., tokens or coins), known as proof of stake. Thus, decentralization of the distributed ledger allows stakeholders to rely less on a central authority, reduces the likelihood of a single corrupt node to cause the entire network to crash, and prevents fraud.

Interaction with a block generally relies on asymmetric cryptography between users identified by a user identification number, rather than a name. Asymmetric cryptography uses key pairs for encryption, a private key, which through encryption yields an associated public key. In one non-limiting example, the first asymmetric key scheme created is called RSA, wherein two large prime numbers comprise the private key and the product of these two large prime numbers is the public key. Thus, new applications of the distributed blockchain database enable secure transactions based on publically defined rules, while ensuring a level of anonymity.

Cryptocurrency platforms utilize tokens, or coins, as currencies, that are used to facilitate the distribution and sharing of rewards and benefits to its stakeholders. Since tokens held in distributed ledgers on a blockchain are virtually impossible to alter and the terms of their issue (e.g., supply) are usually well defined, the ability to manage the token supply for the respective token ecosystem has proven to be problematic.

If the token ecosystem expands rapidly, the growing appreciation of the price of a token requires more tokens to be issued to stabilize the price. Failure to issue more tokens results in a token that significantly appreciates in price. One of the hallmarks of cryptocurrencies, like Bitcoin, is the finite supply, which guarantees significant appreciation of the price as the availability of tokens diminishes. While the significant appreciation in a token price is welcomed by speculative token investors, platforms utilizing blockchain to track and incentive human behavior, rely on a stable token price to facilitate future transactions between buyers who award the tokens to constituents, and the constituents that earn the tokens and redeem them.

Thus, there is a long-felt unmet need for a secure and decentralized utility token system built on a blockchain platform that is insulated from the significant fluctuations in token price. Such a utility token system would be particularly suited to track and incentivize healthy human behavior, such as eating nutritious food and exercising.

One advantage of the present disclosure is the ability to track user compliance with a health program through an immutable, distributed ledger of transactions (e.g., the blockchain). Transactions are stored on a sequence of blocks on the blockchain in a token ecosystem, in which tokens are used to track and incentivize user compliance with health-related goals. A hashing function is used to generate a unique hash for the contents of each block, which can be stored on the block and a next block, thus linking the blocks via their hashes. The decentralized nature of the nodes allows a consensus to be achieved for a new block added to the blockchain without relying on a single, centralized authority that may be vulnerable to hacking.

Another advantage of the present disclosure is the ability to stabilize the price of tokens in a supply of tokens provided in an ecosystem, such as the ecosystem disclosed, enabling stakeholders the ability to make decisions efficiently regarding future transactions. The methods for stabilizing the price of the tokens provide significant advantages over known methods that rely on complex algorithms to calculate how many tokens to add to, or subtract from, the ecosystem.

One aspect provided herein is a utility token system, comprising: a distributed ledger; a back-end system; and a digital wallet associated with a key address, the digital wallet configured to: provide, to the back-end system, a first transaction comprising a recipient and a token from a plurality of tokens, the token associated with the key address; the back-end system configured to: generate a smart contract associated with the tokens, the smart contract comprising a set of instructions which, when invoked with the first transaction and executed, by one or more processors, cause the one or more processors to perform operations comprising: determining a current supply of the tokens based on a multiplication block stored to the distributed ledger, wherein the token represents a portion of the current supply of the tokens; determining a valuation of the token based on the current supply of the tokens; providing the valuation of the token to the recipient; persist the smart contract to the distributed ledger; and when receiving the first transaction from the digital wallet, invoking the smart contract with the first transaction.

In some embodiments, the back-end system is configured to: receive the multiplication block signed by a key associated with a central authority; verify the signature based on a public address of the key; persist the multiplication block to the distributed ledger. In some embodiments, the system comprises a peer-to-peer network, the peer-to-peer network comprising a plurality of nodes, wherein the multiplication block is verified by a plurality of the nodes. In some embodiments, persisting the multiplication block generates a hard fork of the distributed ledger. In some embodiments, the current supply of the tokens is determined based on an off-chain oracle instead of the multiplication block. In some embodiments, the back-end system is configured to: receive, from an affiliate vendor, a second transaction associating the token with the key address; generate a block comprising the second transaction; and persist the block to the distributed ledger. In some embodiments, the system comprises a peer-to-peer network, the peer-to-peer network comprising a plurality of nodes, wherein the block is verified by a plurality of the nodes. In some embodiments, the multiplication block comprises a multiplier for the number of tokens. In some embodiments, the multiplier is determined based on a reference price for each of the tokens. In some embodiments, the reference price is predetermined. In some embodiments, the reference price comprises an initial price for the tokens. In some embodiments, the reference price mirrors a price of an existing currency or cryptocurrency. In some embodiments, the multiplier comprises a decimal value or a fractional value. In some embodiments, the recipient comprises an affiliate vendor, and wherein the valuation of the token determines a discount, rebate, or coupon for the affiliate vendor. In some embodiments, the system comprises a second digital wallet associated with a second key address, wherein the digital wallet is configured to: provide a transfer transaction comprising the token and the second key address; the back-end system configured to: when receiving the transfer transaction from the digital wallet, generate a transfer block comprising the transfer transaction; and persist the transfer block to the distributed ledger. In some embodiments, the system comprises a peer-to-peer network, the peer-to-peer network comprising a plurality of nodes, wherein the transfer block is verified by a plurality of the nodes.

In some aspects, disclosed herein are methods of stabilizing the price of tokens in a supply of tokens provided in an ecosystem. In some embodiments, the price of tokens decreases, when a token in the supply of tokens is split into more than one token (hereinafter referred to as “token-split”). In some embodiments, the price of tokens increases, when a token in the supply of tokens is reverse-split into less than one token (hereinafter referred to as “reverse-split”). Further provided are ecosystems, systems, networks, media, and methods utilizing a peer-to-peer decentralized distributed ledger computer network to track user compliance with a health program. In some embodiments, the distributed ledger computer network comprises a blockchain, leveraging tokens to track user transactions within the ecosystem. In some embodiments, the price of the tokens in the supply of tokens provided to the ecosystem is stabilized using the methods disclosed, to ensure efficient decision making regarding future transactions between stakeholders in the ecosystem.

Aspects disclosed herein provide a computer-implemented method of tracking user compliance with a health program, comprising: a. storing, in a personal health database at a personal health tracking system, a profile associated with an account belonging to a user, wherein the profile comprises goals related to health; b. offering a token to the user, wherein the token represents an encrypted data structure that is processed and stored in a distributed ledger computer network and is redeemable by the user at an affiliate vendor or a health entity in exchange for goods or services provided by the affiliate vendor or health entity; c. receiving at least one data unit from the affiliate vendor or health entity comprising information about a transaction between the user and the affiliate vendor or the health entity, the transaction comprising (i) the user earning the token as a reward for healthy behavior or (ii) the user redeeming the token at the affiliate vendor or health entity for the goods or services, where the goods or services assist the user in meeting the goals related to health; d. processing the at least one data unit to generate an encrypted data structure and store the encrypted data structure; e. generating a hash of the at least one data unit using a cryptographic hash function; f publishing a new transaction comprising the hash of the data to the distributed ledger computing network for validation and addition to the distributed ledger computing network; and g. selling the token. In some embodiments, affiliate vendor comprises a grocery store, gym, or virtual fitness tracking company. In some embodiments, the goals are determined by the user, the affiliate vendor, or the health entity, or a combination thereof. In some embodiments, the at least one data unit comprises structured data. In some embodiments, the information about the transaction comprises the date, time, parties, and value exchanged in the transaction. In some embodiments, the method further comprises: a. storing in a health entity database at the personal health tracking system, a profile associated with an account belonging to the health entity, wherein the profile comprises goals for the user related to the health of the user; b. offering a token to the health entity; and c. optionally, holding the token for the health entity until user compliance with the goal related to health is determined to be completed by the user. In some embodiments, the method further comprises: a. storing in an affiliate vendor database at the personal health tracking system, a profile associated with an account belonging to the affiliated vendor, wherein the profile comprises goals for the user related to health of the user; b. offering a token to the affiliate vendor; and c. optionally, holding the token for the affiliate vendor until user compliance with the goal related to health is determined to be completed by the user. In some embodiments, the health entity comprises a health insurance company, an employer, a non-profit charity, a government, or entities sponsored, at least in part, by the health insurance company, the employer, the non-profit charity, or the government. In some embodiments, the token is configured to be redeemed at the affiliate vendor or health entity as a coupon, discount, rebate, or any combination thereof. In some embodiments, the token is configured to be sold to a third party. In some embodiments, the user sells or gives the token to an entity that is not an affiliate vendor or health entity. In some embodiments, the method further comprises: a. providing a supply of tokens; b. integrating into the distributed ledger computer network an off-chain oracle configured to expand or contract the supply of tokens based on a reference price; and b. multiplying the supply of tokens by a number greater than one to expand the supply of tokens if the price of a token exceeds the reference price, or multiplying the supply of tokens by a number less than one to contract the supply of tokens if the price of a token is below the reference price. In some embodiments, the method further comprises: a. providing a special purpose central authority private key configured to expand or contract the supply of tokens; and b. multiplying the supply of tokens by a number greater than one to expand the supply of tokens, or multiplying the supply of tokens by a number less than one to contract the supply of tokens. In some embodiments, the supply of tokens expands by a single token becoming more than one token and the supply of tokens contracts by a single token becoming less than one token. In some embodiments, the off-chain oracle is not configured to expand the supply of tokens with the addition of more tokens, and wherein the off-chain oracle is not configured to contract the supply of tokens with the reduction of tokens. In some embodiments, the number greater than one comprises a decimal or fraction. In some embodiments, the number less than one comprises a decimal or fraction. In some embodiments, the reference price is predetermined. In some embodiments, the reference price comprises the initial token price. In some embodiments, the reference price mirrors a price of an existing currency or cryptocurrency. In some embodiments, the supply of tokens is expanded by the off-chain oracle if the price of a token exceeds the price of the reference price by two percent, three percent, four percent, five percent, six percent, seven percent, eight percent, nine percent, or ten percent. In some embodiments, the supply of tokens is contracted by the off-chain oracle if the price of a token is below the price of the reference price by two percent, three percent, four percent, five percent, six percent, seven percent, eight percent, nine percent, or ten percent. In some embodiments, the special purpose central authority private key is provided by a central authority that determines whether the supply of tokens should be expanded or contracted. In some embodiments, the central authority does not have authority to expand the supply of tokens with the addition of more tokens, and wherein the central authority does not have authority to contract the supply of tokens with the reduction of tokens. In some embodiments, the token is not bitcoin. In some embodiments, the distributed ledger computer network is published on a private decentralized peer-to-peer network. In some embodiments, the distributed ledger computer network is published on a public decentralized peer-to-peer network. In various exemplary embodiments, the distributed ledger computer network utilizes a blockchain platform comprising Ethereum, Zcash, Litecoin, Namecoin, Swiftcoin, Bytecoin, Peercoin, Emercoin, Feathercoin, Gridcoin, Primecoin, Ripple, Auroracoin, Dash, Zetacoin, Monero, Tether, Mazacoin, or Vertcoin.

Aspects disclosed herein provide a computer-implemented methods of stabilizing the price of a utility token, comprising: a. providing a supply of tokens, wherein each token represents an encrypted data structure that is processed and stored in a distributed ledger computer network, the tokens configured to be used by the user to purchase goods or services from an affiliate vendor or health entity; b. receiving at least one data unit comprising information about a transaction; c. processing the at least one data unit to generate an encrypted data structure and store the encrypted data structure; d. generating a hash of the at least one data unit using a cryptographic hash function; e. publishing a new transaction comprising the hash of the data to the distributed ledger computing network for validation and addition to the distributed ledger computer network; f. providing a special purpose central authority private key configured to expand or contract the supply of tokens; and g. multiplying the supply of tokens by a number greater than one to expand the supply of tokens or multiplying the supply of tokens by a number less than one to contract the supply of tokens. In some embodiments, the transaction comprises (i) the user earning the token as a reward for healthy behavior or (ii) the user redeeming the token at an affiliate vendor or health entity for the goods or services provided by the affiliate vendor or health entity. In some embodiments, the affiliate vendor comprises a grocery store, gym, or virtual fitness tracking company. In some embodiments the at least one data unit comprises structured data. In some embodiments the information about the transaction comprises the date, time, parties, and value exchanged in the transaction. In some embodiments, the method further comprises: a. storing in a personal health database at a personal health tracking system, a profile associated with an account belonging to the user, wherein the profile comprises goals related to health; and b. offering a token to the user. In some embodiments, the method further comprises: a. storing in a health entity database at a personal health tracking system, a profile associated with an account belonging to the health entity, wherein the profile comprises goals for the user related to the health of the user; b. offering a token to the health entity; and c. optionally, holding the token for a health entity until user compliance with the goal related to health is determined to be completed by the user. In some embodiments, the method further comprises: a. storing in an affiliate vendor database at the personal health tracking system, a profile associated with an account belonging to the affiliated vendor, wherein the profile comprises goals for the user related to health of the user; b. offering a token to the health entity; and c. optionally, holding the token for the health entity until user compliance with the goal related to health is determined to be completed by the user. In some embodiments, the health entity comprises a health insurance company, an employer, a non-profit charity, a government, or entities sponsored, at least in part, by the health insurance company, the employer, the non-profit charity, or the government. In some embodiments, the token is configured to be redeemed at the affiliate vendor or health entity as a coupon, discount, rebate, or any combination thereof. In some embodiments, the token is configured to be sold to a third party. In some embodiments, the distributed ledger computer network is published on a private decentralized peer-to-peer network. In some embodiments, the distributed ledger computer network is published on a public decentralized peer-to-peer network. In various exemplary embodiments, the distributed ledger computer network utilizes a blockchain platform comprising Ethereum, Zcash, Litecoin, Namecoin, Swiftcoin, Bytecoin, Peercoin, Emercoin, Feathercoin, Gridcoin, Primecoin, Ripple, Auroracoin, Dash, Zetacoin, Monero, Tether, Mazacoin, or Vertcoin. In some embodiments, the supply of tokens expands by a single token becoming more than one token and the supply of tokens contracts by a single token becoming less than one token. In some embodiments, the central authority does not have authority to expand the supply of tokens with the addition of more tokens, and wherein the central authority does not have authority to contract the supply of tokens with the reduction of tokens. In some embodiments, the number greater than one comprises a decimal or fraction. In some embodiments, the number less than one comprises a decimal or fraction.

Aspects disclosed herein provide a computer-implemented method of stabilizing the price of a utility token, comprising: a. providing a supply of tokens, wherein each token represents an encrypted data structure that is processed and stored in a distributed ledger computer network, the tokens configured to be used by the user to purchase goods or services from an affiliate vendor or health entity; b. receiving at least one data unit comprising information about a transaction; c. processing the at least one data unit to generate an encrypted data structure and store the encrypted data structure; d. generating a hash of the at least one data unit using a cryptographic hash function; e. publishing a new transaction comprising the hash of the data to the distributed ledger computing network for validation and addition to the distributed ledger computer network; f. integrating into the distributed ledger computer network an off-chain oracle configured to expand or contract the supply of tokens based on a reference price; and g. multiplying the supply of tokens by a number greater than one to expand the supply of tokens if the price of a token exceeds the reference price, or multiplying the supply of tokens by a number less than one to contract the supply of tokens if the price of a token is below the reference price. In some embodiments, the transaction comprises (i) the user earning the token as a reward for healthy behavior or (ii) the user redeeming the token at an affiliate vendor or health entity for the goods or services provided by the affiliate vendor or health entity. In some embodiments, the affiliate vendor comprises a grocery store, gym, or virtual fitness tracking company. In some embodiments, the at least one data unit comprises structured data. In some embodiments, the information about the transaction comprises the date, time, parties, and value exchanged in the transaction. In some embodiments, the method further comprises: a. storing in a personal health database at a personal health tracking system, a profile associated with an account belonging to the user, wherein the profile comprises goals related to health; and b. offering a token to the user. In some embodiments, the method further comprises: a. storing in a health entity database at the personal health tracking system, a profile associated with an account belonging to the health entity, wherein the profile comprises goals for the user related to the health of the user; b. offering a token to the health entity; and c. optionally, holding the token for the health entity until user compliance with the goal related to health is determined to be completed by the user. In some embodiments, the method further comprises: a. storing in an affiliate vendor database at the personal health tracking system, a profile associated with an account belonging to the affiliated vendor, wherein the profile comprises goals for the user related to health of the user; b. offering a token to the affiliate vendor; and c. optionally, holding the token for the affiliate vendor until user compliance with the goal related to health is determined to be completed by the user. In some embodiments, the health entity comprises a health insurance company, an employer, a non-profit charity, a government, or entities sponsored, at least in part, by the health insurance company, the employer, the non-profit charity, or the government. In some embodiments, the token is configured to be redeemed at the affiliate vendor or health entity as a coupon, discount, or rebate, or any combination thereof. In some embodiments, the token is configured to be sold to a third party. In some embodiments, the distributed ledger computer network is published on a private decentralized peer-to-peer network. In some embodiments, the distributed ledger computer network is published on a public decentralized peer-to-peer network. In various exemplary embodiments, the distributed ledger computer network utilizes a blockchain platform comprising Ethereum, Zcash, Litecoin, Namecoin, Swiftcoin, Bytecoin, Peercoin, Emercoin, Feathercoin, Gridcoin, Primecoin, Ripple, Auroracoin, Dash, Zetacoin, Monero, Tether, Mazacoin, or Vertcoin. In some embodiments, the reference price is predetermined. In some embodiments, the reference price comprises the initial token price. In some embodiments, the reference price mirrors a price of an existing currency or cryptocurrency. In some embodiments, the supply of tokens is expanded by the off-chain oracle if the price of a token exceeds the price of the reference price by two percent, three percent, four percent, five percent, six percent, seven percent, eight percent, nine percent, or ten percent. In some embodiments, the supply of tokens is contracted by the off-chain oracle if the price of a token is below the price of the reference price by two percent, three percent, four percent, five percent, six percent, seven percent, eight percent, nine percent, or ten percent. In some embodiments, the supply of tokens expands by a single token becoming more than one token and the supply of tokens contracts by a single token becoming less than one token. In some embodiments, the off-chain oracle is not configured to expand the supply of tokens with the addition of more tokens, and wherein the off-chain oracle is not configured to contract the supply of tokens with the reduction of tokens. In some embodiments, the number greater than one comprises a decimal or fraction. In some embodiments, the number less than one comprises a decimal or fraction.

Aspects disclosed herein provide a non-transitory computer readable storage medium, comprising computer-executable code configured to cause at least one processor to: a. store, in a personal health database at a personal health tracking system, a profile associated with an account belonging to a user, wherein the profile comprises goals related to health; b. offer a token to the user, wherein the token represents an encrypted data structure that is processed and stored in a distributed ledger computer network and is redeemable by the user at an affiliate vendor or a healthy entity in exchange for goods or services provided by the affiliate vendor or health entity; c. receive at least one data unit from the affiliate vendor or health entity comprising information about a transaction between the user and the affiliate vendor or the health entity, the transaction comprising (i) the user earning the token as a reward for healthy behavior or (ii) the user redeeming the token at the affiliate vendor or health entity for the goods or services, the goods or services assist the user in meeting the goals related to health; d. process the at least one data unit to generate an encrypted data structure and store the encrypted data structure; e. generate a hash of the at least one data unit using a cryptographic hash function; f. publish a new transaction comprising the hash of the data to the distributed ledger computing network for validation and addition to the distributed ledger computing network; and g. sell the token. In some embodiments, the affiliate vendor comprises a grocery store, gym, or virtual fitness tracking company. In some embodiments, the goals are determined by the user, the affiliate vendor, or the health entity, or a combination thereof. In some embodiments, the at least one data unit comprises structured data. In some embodiments, the information about the transaction comprises the date, time, parties, and value exchanged in the transaction. In some embodiments, the computer-executable code configured to cause at least one processor to: a. store in a health entity database at the personal health tracking system, a profile associated with an account belonging to the health entity, wherein the profile comprises goals for the user related to the health of the user; b. offer a token to the health entity; and c. optionally, hold the token for the health entity until user compliance with the goal related to health is determined to be completed by the user. In some embodiments, the computer-executable code configured to cause at least one processor to: a. store in an affiliate vendor database at the personal health tracking system, a profile associated with an account belonging to the affiliated vendor, wherein the profile comprises goals for the user related to health of the user; b. offer a token to the health entity; and c. optionally, hold the token for the health entity until user compliance with the goal related to health is determined to be completed by the user. In some embodiments, the health entity comprises a health insurance company, an employer, a non-profit charity, a government, or entities sponsored, at least in part, by the health insurance company, the employer, the non-profit charity, or the government. In some embodiments, the token is configured to be redeemed at the affiliate vendor or health entity as a coupon, discount, rebate, or any combination thereof. In some embodiments, the token is configured to be sold to a third party. In some embodiments, the computer-executable code configured to cause at least one processor to: a. provide a supply of tokens; b. integrate into the distributed ledger computer networker network an off-chain oracle configured to expand or contract the supply of tokens based on a reference price; and b. multiply the supply of tokens by a number greater than one to expand the supply of tokens if the price of a token exceeds the reference price, or multiplying the supply of tokens by a number less than one to contract the supply of tokens if the price of a token is below the reference price. In some embodiments, the computer-executable code configured to cause at least one processor to: a. provide a special purpose central authority private key configured to expand or contract the supply of tokens; and b. multiply the supply of tokens by a number greater than one to expand the supply of tokens, or multiplying the supply of tokens by a number less than one to contract the supply of tokens. In some embodiments, the off-chain oracle is not configured to expand the supply of tokens with the addition of more tokens, and wherein the off-chain oracle is not configured to contract the supply of tokens with the reduction of tokens. In some embodiments, the central authority does not have authority to expand the supply of tokens with the addition of more tokens, and wherein the central authority does not have authority to contract the supply of tokens with the reduction of tokens. In some embodiments, the number greater than one comprises a decimal or fraction. In some embodiments, the number less than one comprises a decimal or fraction. In some embodiments, the reference price is predetermined. In some embodiments the reference price comprises the initial token price. In some embodiments, the reference price mirrors a price of an existing currency or cryptocurrency. In some embodiments, the supply of tokens is expanded by the off-chain oracle if the price of a token exceeds the price of the reference price by two percent, three percent, four percent, five percent, six percent, seven percent, eight percent, nine percent, or ten percent. In some embodiments, the supply of tokens is contracted by the off-chain oracle if the price of a token is below the price of the reference price by two percent, three percent, four percent, five percent, six percent, seven percent, eight percent, nine percent, or ten percent. In some embodiments, the token is not bitcoin. In some embodiments, the distributed ledger computer network is published on a private decentralized peer-to-peer network. In some embodiments, the distributed ledger computer network is published on a public decentralized peer-to-peer network. In various exemplary embodiments, the distributed ledger computer network utilizes a blockchain platform comprising Ethereum, Zcash, Litecoin, Namecoin, Swiftcoin, Bytecoin, Peercoin, Emercoin, Feathercoin, Gridcoin, Primecoin, Ripple, Auroracoin, Dash, Zetacoin, Monero, Tether, Mazacoin, or Vertcoin. In some embodiments, the supply of tokens expands by a single token becoming more than one token and the supply of tokens contracts by a single token becoming less than one token.

Aspects disclosed herein provide a non-transitory computer readable storage medium, comprising computer-executable code configured to cause at least one processor to: a. provide a supply of tokens, wherein each token represents an encrypted data structure that is processed and stored in a distributed ledger computer network, the tokens configured to be used by the user to purchase goods or services from an affiliate vendor or health entity; b. receive at least one data unit comprising information about a transaction; c. process the at least one data unit to generate an encrypted data structure and store the encrypted data structure; d. generate a hash of the at least one data unit using a cryptographic hash function; e. publish a new transaction comprising the hash of the data to the distributed ledger computing network for validation and addition to the distributed ledger computer network; f provide a special purpose central authority private key configured to expand or contract the supply of tokens; and g. multiply the supply of tokens by a number greater than one to expand the supply of tokens or multiplying the supply of tokens by a number less than one to contract the supply of tokens. In some embodiments, the transaction comprises (i) the user earning the token as a reward for healthy behavior or (ii) the user redeeming the token at an affiliate vendor or health entity for the goods or services provided by the affiliate vendor or health entity. In some embodiments, the affiliate vendor comprises a grocery store, gym, or virtual fitness tracking company. In some embodiments, the at least one data unit comprises structured data. In some embodiments, the information about the transaction comprises the date, time, parties, and value exchanged in the transaction. In some embodiments, the computer-executable code configured to cause at least one processor to: a. store in a personal health database at a personal health tracking system, a profile associated with an account belonging to the user, wherein the profile comprises goals related to health; and b. offer a token to the user. In some embodiments, the computer-executable code configured to cause at least one processor to: a. store in a health entity database at a personal health tracking system, a profile associated with an account belonging to the health entity, wherein the profile comprises goals for the user related to the health of the user; b. offer a token to the health entity; and c. optionally, hold the token for the health entity until user compliance with the goal related to health is determined to be completed by the user. In some embodiments, the computer-executable code configured to cause at least one processor to: a. store in an affiliate vendor database at a personal health tracking system, a profile associated with an account belonging to the affiliated vendor, wherein the profile comprises goals for the user related to health of the user; b. offer a token to the affiliate vendor; and c. optionally, hold the token for the affiliate vendor until user compliance with the goal related to health is determined to be completed by the user. In some embodiments, the health entity comprises a health insurance company, an employer, a non-profit charity, a government, or entities sponsored, at least in part, by the health insurance company, the employer, the non-profit charity, or the government. In some embodiments, the token is configured to be redeemed at the affiliate vendor or health entity as a coupon, discount, rebate, or any combination thereof. In some embodiments, the token is configured to be sold to a third party. In some embodiments, the distributed ledger computer network is published on a private decentralized peer-to-peer network. In some embodiments, the distributed ledger computer network is published on a public decentralized peer-to-peer network. In various exemplary embodiments, the distributed ledger computer network utilizes a blockchain platform comprising Ethereum, Zcash, Litecoin, Namecoin, Swiftcoin, Bytecoin, Peercoin, Emercoin, Feathercoin, Gridcoin, Primecoin, Ripple, Auroracoin, Dash, Zetacoin, Monero, Tether, Mazacoin, or Vertcoin. In some embodiments, the supply of tokens expands by a single token becoming more than one token and the supply of tokens contracts by a single token becoming less than one token. In some embodiments, the central authority does not have authority to expand the supply of tokens with the addition of more tokens, and wherein the central authority does not have authority to contract the supply of tokens with the reduction of tokens. In some embodiments, the number greater than one comprises a decimal or fraction. In some embodiments, the number less than one comprises a decimal or fraction.

Aspects disclosed herein provide a non-transitory computer readable storage medium, comprising computer-executable code configured to cause at least one processor to: a. provide a supply of tokens, wherein each token represents an encrypted data structure that is processed and stored in a distributed ledger computer network, the tokens configured to be used by the user to purchase goods or services from an affiliate vendor or health entity; b. receive at least one data unit comprising information about a transaction; c. process the at least one data unit to generate an encrypted data structure and store the encrypted data structure; d. generate a hash of the at least one data unit using a cryptographic hash function; e. publish a new transaction comprising the hash of the data to the distributed ledger computing network for validation and addition to the distributed ledger computer network; f integrate into the distributed ledger computer network an off-chain oracle configured to expand or contract the supply of tokens based on a reference price; and g. multiply the supply of tokens by a number greater than one to expand the supply of tokens if the price of a token exceeds the reference price, or multiplying the supply of tokens by a number less than one to contract the supply of tokens if the price of a token is below the reference price. In some embodiments, the transaction comprises (i) the user earning the token as a reward for healthy behavior or (ii) the user redeeming the token at an affiliate vendor or health entity for the goods or services provided by the affiliate vendor or health entity. In some embodiments, the affiliate vendor comprises a grocery store, gym, or virtual fitness tracking company. In some embodiments, the at least one data unit comprises structured data. In some embodiments, the information about the transaction comprises the date, time, parties, and value exchanged in the transaction. In some embodiments, the computer-executable code configured to cause at least one processor to: a. store in a personal health database at a personal health tracking system, a profile associated with an account belonging to the user, wherein the profile comprises goals related to health; and b. offer a token to the user. In some embodiments, the computer-executable code configured to cause at least one processor to: a. store in a health entity database at a personal health tracking system, a profile associated with an account belonging to the health entity, wherein the profile comprises goals for the user related to the health of the user; b. offer a token to the health entity; and c. optionally, hold the token for the health entity until user compliance with the goal related to health is determined to be completed by the user. In some embodiments, the computer-executable code configured to cause at least one processor to: a. store in an affiliate vendor database at a personal health tracking system, a profile associated with an account belonging to the affiliated vendor, wherein the profile comprises goals for the user related to health of the user; b. offer a token to the affiliate vendor; and c. optionally, hold the token for the affiliate vendor until user compliance with the goal related to health is determined to be completed by the user. In some embodiments, the health entity comprises a health insurance company, an employer, a non-profit charity, a government, or entities sponsored, at least in part, by the health insurance company, the employer, the non-profit charity, or the government. In some embodiments, the token is configured to be redeemed at the affiliate vendor or health entity as a coupon, discount, rebate, or any combination thereof. In some embodiments, the token is configured to be sold to a third party. In some embodiments, the price of a token does not change according to supply and demand. In some embodiments, the distributed ledger computer network is published on a private decentralized peer-to-peer network. In some embodiments, the distributed ledger computer network is published on a public decentralized peer-to-peer network. In various exemplary embodiments, the distributed ledger computer network utilizes a blockchain platform comprising Ethereum, Zcash, Litecoin, Namecoin, Swiftcoin, Bytecoin, Peercoin, Emercoin, Feathercoin, Gridcoin, Primecoin, Ripple, Auroracoin, Dash, Zetacoin, Monero, Tether, Mazacoin, or Vertcoin. In some embodiments, the reference price is predetermined. In some embodiments, the reference price comprises the initial token price. In some embodiments, the reference price mirrors a price of an existing currency or cryptocurrency. In some embodiments, the supply of tokens is expanded by the off-chain oracle if the price of a token exceeds the price of the reference price by two percent, three percent, four percent, five percent, six percent, seven percent, eight percent, nine percent, or ten percent. The non-transitory computer readable storage medium of claim 111, wherein the supply of tokens is contracted by the off-chain oracle if the price of a token is below the price of the reference price by two percent, three percent, four percent, five percent, six percent, seven percent, eight percent, nine percent, or ten percent. In some embodiments, the supply of tokens expands by a single token becoming more than one token and the supply of tokens contracts by a single token becoming less than one token. In some embodiments, the off-chain oracle is not configured to expand the supply of tokens with the addition of more tokens, and wherein the off-chain oracle is not configured to contract the supply of tokens with the reduction of tokens. In some embodiments, the number greater than one comprises a decimal or fraction. In some embodiments, the number less than one comprises a decimal or fraction.

Aspects disclosed herein provide a distributed ledger computer network for tracking and incentivizing user compliance with a health program, the network comprising: a. a plurality of computing devices, each computing device comprising at least one processor, a memory, and an operating system, wherein a computing device from the plurality of computing devices is configured to: i. store, in a personal health database at a personal health tracking system, a profile associated with an account belonging to a user, wherein the profile comprises goals related to health; ii. offer a token to the user, wherein the token represents an encrypted data structure that is processed and stored in a distributed ledger computer network and is redeemable by the user at an affiliate vendor or a health entity in exchange for goods or services provided by the affiliate vendor or health entity; iii. receive at least one data unit from the affiliate vendor or health entity comprising information about a transaction between the user and the affiliate vendor or the health entity, the transaction comprising the (i) the user earning the token as a reward for healthy behavior or (ii) user redeeming the token at the affiliate vendor or health entity for the goods or services, where the goods or services assist the user in meeting the goals related to health; iv. process the at least one data unit to generate an encrypted data structure and store the encrypted data structure; v. generate a hash of the at least one data unit using a cryptographic hash function; vi. publish a new transaction comprising the hash of the data to the distributed ledger computing network for validation and addition to the distributed ledger computing network; and vii. sell the token. In some embodiments, the affiliate vendor comprises a grocery store, gym, or virtual fitness tracking company. In some embodiments, the goals are determined by the user, the affiliate vendor, or the health entity, or a combination thereof. In some embodiments, the at least one at least one data unit comprises structured data. In some embodiments, the information about the transaction comprises the date, time, parties, and value exchanged in the transaction. In some embodiments, the computing device is further configured to: i. store in a health entity database at the personal health tracking system, a profile associated with an account belonging to the health entity, wherein the profile comprises goals for the user related to the health of the user; ii. offer a token to the health entity; and iii. optionally, hold the token for the health entity until user compliance with the goal related to health is determined to be completed by the user. In some embodiments, the computing device is further configured to: i. store in an affiliate vendor database at the personal health tracking system, a profile associated with an account belonging to the affiliated vendor, wherein the profile comprises goals for the user related to health of the user; ii. offer a token to the health entity; and iii. optionally, hold the token for the health entity until user compliance with the goal related to health is determined to be completed by the user. In some embodiments, the health entity comprises a health insurance company, an employer, a non-profit charity, a government, or entities sponsored, at least in part, by the health insurance company, the employer, the non-profit charity, or the government. In some embodiments, the token is configured to be redeemed at the affiliate vendor or health entity as a coupon, discount, rebate, or any combination thereof. In some embodiments, the token is configured to be sold to a third party. In some embodiments, the computing device is further configured to: i. integrate into the distributed ledger computer network an off-chain oracle configured to expand or contract the supply of tokens based on a reference price; and ii. multiply the supply of tokens by a number greater than one to expand the supply of tokens if the price of a token exceeds the reference price, or multiplying the supply of tokens by a number less than one to contract the supply of tokens if the price of a token is below the reference price. In some embodiments, the computing device is further configured to: i. provide a special purpose central authority private key configured to expand or contract the supply of tokens; and ii. multiply the supply of tokens by a number greater than one to expand the supply of tokens, or multiplying the supply of tokens by a number less than one to contract the supply of tokens. In some embodiments, the reference price is predetermined. In some embodiments, the reference price comprises the initial token price. In some embodiments, the reference price mirrors a price of an existing currency or cryptocurrency. In some embodiments, the off-chain oracle is not configured to expand the supply of tokens with the addition of more tokens, and wherein the off-chain oracle is not configured to contract the supply of tokens with the reduction of tokens. In some embodiments, the number greater than one comprises a decimal or fraction. In some embodiments, the number less than one comprises a decimal or fraction. In some embodiments, the supply of tokens is expanded by the off-chain oracle if the price of a token exceeds the price of the reference price by two percent, three percent, four percent, five percent, six percent, seven percent, eight percent, nine percent, or ten percent. In some embodiments, the supply of tokens is contracted by the off-chain oracle if the price of a token is below the price of the reference price by two percent, three percent, four percent, five percent, six percent, seven percent, eight percent, nine percent, or ten percent. In some embodiments, the token is not bitcoin. In some embodiments, the distributed ledger computer network is published on a private decentralized peer-to-peer network. In some embodiments, the distributed ledger computer network is published on a public decentralized peer-to-peer network. In various exemplary embodiments, the distributed ledger computer network utilizes a blockchain platform comprising Ethereum, Zcash, Litecoin, Namecoin, Swiftcoin, Bytecoin, Peercoin, Emercoin, Feathercoin, Gridcoin, Primecoin, Ripple, Auroracoin, Dash, Zetacoin, Monero, Tether, Mazacoin, or Vertcoin. In some embodiments, the supply of tokens expands by a single token becoming more than one token and the supply of tokens contracts by a single token becoming less than one token. In some embodiments, the special purpose central authority private key is provided by a central authority that determines whether the supply of tokens should be expanded or contracted. In some embodiments, the central authority does not have authority to expand the supply of tokens with the addition of more tokens, and wherein the central authority does not have authority to contract the supply of tokens with the reduction of tokens.

Aspects disclosed herein provide a personal health tracking system comprising: a. a network interface configured to communicate via a communication network; and b. a computing device in communication with a plurality of computing devices via a distributed ledger computing network, the computing device comprising at least one processor, a memory, and a computer program including instructions executable by the at least one processor to track user compliance with goals related to health, the instructions comprising: i. storing, in a personal health database at a personal health tracking system, a profile associated with an account belonging to a user; ii. providing to the computing device an application configured to communicate with the personal healthy tracking system; iii. providing a supply of tokens, wherein each token represents an encrypted data structure that is processed and stored in a distributed ledger computer network, the tokens configured to be used by the user to purchase goods or services from an affiliate vendor or health entity; iv. offering a token to the user; v. receiving at least one data unit comprising information about a transaction; vi. processing the at least one data unit to generate an encrypted data structure and store the encrypted data structure; vi. generating a hash of the at least one data unit using a cryptographic hash function; vii. publishing a new transaction comprising the hash of the data to the distributed ledger computing network for validation and addition to the distributed ledger computer network; viii. integrating into the distributed ledger computer network an off-chain oracle configured to expand or contract the supply of tokens based on a reference price; and ix. multiplying the supply of tokens by a number greater than one to expand the supply of tokens if the price of a token exceeds the reference price, or multiplying the supply of tokens by a number less than one to contract the supply of tokens if the price of a token is below the reference price. In some embodiments, the transaction comprises (i) the user earning the token as a reward for healthy behavior or (ii) the user redeeming the token at an affiliate vendor or health entity for the goods or services provided by the affiliate vendor or health entity. In some embodiments, the affiliate vendor comprises a grocery store, gym, or virtual fitness tracking company. In some embodiments, the goals are determined by the user, the affiliate vendor, or the health entity, or a combination thereof. In some embodiments, the at least one data unit comprises structured data. In some embodiments, the information about the transaction comprises the date, time, parties, and value exchanged in the transaction. In some embodiments, the instructions further comprise: i. storing in a health entity database at the personal health tracking system, a profile associated with an account belonging to the health entity, wherein the profile comprises goals for the user related to the health of the user; ii. offering a token to the health entity; and iii. optionally, holding the token for the health entity until user compliance with the goal related to health is determined to be completed by the user. In some embodiments, the instructions further comprise: i. storing in an affiliate vendor database at the personal health tracking system, a profile associated with an account belonging to the affiliated vendor, wherein the profile comprises goals for the user related to health of the user; ii. offering a token to the affiliate vendor; and iii. optionally, holding the token for the affiliate vendor until user compliance with the goal related to health is determined to be completed by the user. In some embodiments, the health entity comprises a health insurance company, an employer, a non-profit charity, a government, or entities sponsored, at least in part, by the health insurance company, the employer, the non-profit charity, or the government. In some embodiments, the token is configured to be redeemed at the affiliate vendor or health entity as a coupon, discount, rebate, or any combination thereof. In some embodiments, the distributed ledger computer network is published on a private decentralized peer-to-peer network. In some embodiments, the distributed ledger computer network is published on a public decentralized peer-to-peer network. In various exemplary embodiments, the distributed ledger computer network utilizes a blockchain platform comprising Ethereum, Zcash, Litecoin, Namecoin, Swiftcoin, Bytecoin, Peercoin, Emercoin, Feathercoin, Gridcoin, Primecoin, Ripple, Auroracoin, Dash, Zetacoin, Monero, Tether, Mazacoin, or Vertcoin. In some embodiments, the reference price is predetermined. In some embodiments, the reference price comprises the initial token price. In some embodiments, the reference price mirrors a price of an existing currency or cryptocurrency. In some embodiments, the supply of tokens is expanded by the off-chain oracle if the price of a token exceeds the price of the reference price by two percent, three percent, four percent, five percent, six percent, seven percent, eight percent, nine percent, or ten percent. In some embodiments, the supply of tokens is contracted by the off-chain oracle if the price of a token is below the price of the reference price by two percent, three percent, four percent, five percent, six percent, seven percent, eight percent, nine percent, or ten percent. In some embodiments, the supply of tokens expands by a single token becoming more than one token and the supply of tokens contracts by a single token becoming less than one token. In some embodiments, the off-chain oracle is not configured to expand the supply of tokens with the addition of more tokens, and wherein the off-chain oracle is not configured to contract the supply of tokens with the reduction of tokens. In some embodiments, the number greater than one comprises a decimal or fraction. In some embodiments, the number less than one comprises a decimal or fraction.

Aspects disclosed herein provide a personal health tracking system comprising: a. a network interface configured to communicate via a communication network; and b. a computing device in communication with a plurality of computing devices via a distributed ledger computing network, the computing device comprising at least one processor, a memory, and a computer program including instructions executable by the at least one processor to track user compliance with goals related to health, the instructions comprising: i. storing, in a personal health database at a personal health tracking system, a profile associated with an account belonging to a user; ii. providing to the computing device an application configured to communicate with the personal healthy tracking system; iii. providing a supply of tokens, wherein each token represents an encrypted data structure that is processed and stored in a distributed ledger computer network, the tokens configured to be used by the user to purchase goods or services from an affiliate vendor or health entity; iv. offering a token to the user; v. receiving at least one data unit comprising information about a transaction; vi. processing the at least one data unit to generate an encrypted data structure and store the encrypted data structure; vii. generating a hash of the at least one data unit using a cryptographic hash function; viii. publishing a new transaction comprising the hash of the data to the distributed ledger computing network for validation and addition to the distributed ledger computer network; ix. providing a special purpose central authority private key configured to expand or contract the supply of tokens; and x. multiplying the supply of tokens by a number greater than one to expand the supply of tokens or multiplying the supply of tokens by a number less than one to contract the supply of tokens. In some embodiments, the transaction comprises (i) the user earning the token as a reward for healthy behavior or (ii) the user redeeming the token at an affiliate vendor or health entity for the goods or services provided by the affiliate vendor or health entity. In some embodiments, the affiliate vendor comprises a grocery store, gym, or virtual fitness tracking company. In some embodiments, the goals are determined by the user, the affiliate vendor, or the health entity, or a combination thereof. In some embodiments, the at least one data unit comprises structured data. In some embodiments, the information about the transaction comprises the date, time, parties, and value exchanged in the transaction. In some embodiments, the instructions further comprise: i. storing in a health entity database at the personal health tracking system, a profile associated with an account belonging to the health entity, wherein the profile comprises goals for the user related to the health of the user; ii. offering a token to the health entity; and iii. optionally, holding the token for the health entity until user compliance with the goal related to health is determined to be completed by the user. In some embodiments, the instructions further comprise: i. storing in an affiliate vendor database at the personal health tracking system, a profile associated with an account belonging to the affiliated vendor, wherein the profile comprises goals for the user related to health of the user; ii. offering a token to the health entity; and iii. optionally, holding the token for the health entity until user compliance with the goal related to health is determined to be completed by the user. In some embodiments, the health entity comprises a health insurance company, an employer, a non-profit charity, a government, or entities sponsored, at least in part, by the health insurance company, the employer, the non-profit charity, or the government. In some embodiments, the token is configured to be redeemed at the affiliate vendor or health entity as a coupon, discount, rebate, or any combination thereof. In some embodiments, the token is configured to be sold to a third party. In some embodiments, the distributed ledger computer network is published on a private decentralized peer-to-peer network. In some embodiments, the distributed ledger computer network is published on a public decentralized peer-to-peer network. In various exemplary embodiments, the distributed ledger computer network utilizes a blockchain platform comprising Ethereum, Zcash, Litecoin, Namecoin, Swiftcoin, Bytecoin, Peercoin, Emercoin, Feathercoin, Gridcoin, Primecoin, Ripple, Auroracoin, Dash, Zetacoin, Monero, Tether, Mazacoin, or Vertcoin. In some embodiments, the supply of tokens expands by a single token becoming more than one token and the supply of tokens contracts by a single token becoming less than one token. In some embodiments, the central authority does not have authority to expand the supply of tokens with the addition of more tokens, and wherein the central authority does not have the authority to contract the supply of tokens with the reduction of tokens. In some embodiments, the number greater than one comprises a decimal or fraction. In some embodiments, the number less than one comprises a decimal or fraction.

### BRIEF DESCRIPTION OF THE DRAWINGS

The novel features of aspects provided herein are set forth with particularity in the appended claims. A better understanding of the features and advantages of the present aspects disclosed herein will be obtained by reference to the following detailed description that sets forth illustrative embodiments, in which the principles of the inventive concepts disclosed herein are utilized, and the accompanying drawings (also “Figure” and “FIG.” herein), of which:

FIG. 1 shows an embodiment of the utility token ecosystem;

FIG. 2 shows an embodiment of the personal health tracking system;

FIG. 3 shows a schematic of an aspect of the utility token split or reverse-split process using a central authority;

FIG. 4 shows a schematic of an aspect of the utility token split or reverse-split process using an off-chain oracle;

FIG. 5 shows a flowchart of an example process implemented within the utility token ecosystem;

FIG. 6 shows a non-limiting example of a computing device; in this case, a device with one or more processors, memory, storage, and a network interface;

FIG. 7 shows a non-limiting example of a web/mobile application provision system; in this case, a system providing browser-based and/or native mobile user interfaces; and

FIG. 8 shows a non-limiting example of a cloud-based web/mobile application provision system; in this case, a system comprising an elastically load balanced, auto-scaling web server and application server resources as well synchronously replicated databases.

### DETAILED DESCRIPTION

While various embodiments of the inventive concepts disclosed herein have been shown and described herein, it will be obvious to those skilled in the art that such embodiments are provided by way of example only. Numerous variations, changes, and substitutions may occur to those skilled in the art without departing from the inventive concepts disclosed herein. It should be understood that various alternatives to the embodiments of the inventive concepts disclosed herein described herein may be employed.

As described herein, a distributed ledger computer network, or blockchain technology, is used to track transactions between a user and affiliate vendor, health entity, or both, in a secure decentralized framework that ensures verification of data integrity. When combined with a distributed consensus algorithm, such as “proof or work” or “proof of stake” algorithms, a blockchain can act as a distributed, immutable ledger. This ledger can be used to guarantee the integrity of a piece of data without sharing the data itself. On a P2P network, blockchains storing transaction data have no central points of trust, which allows for the use of decentralized applications and services that no longer depend upon a single party for data verification.

The present disclosure provides non-limiting examples of practical applications of incorporating distributed computing aspects to reward adherence to a health goal or accomplishment of a health milestone with tokens redeemable for a good, a service, or both provided by one or more of an affiliate vendor or a health entity. Further, the present disclosure provides non-limiting examples of practical applications of incorporating distributed computing aspects to convert a measured physical activity (e.g., eating or exercise) to redeemable goods, services, or both provided by one or more of an affiliate vendor or a health entity. In some cases, the distributed computing aspects performed by the methods and systems herein, cannot be performed without one or more non-transitory computer-readable storage media coupled to one or more processors and having instructions stored thereon. In some cases, the distributed computing aspects performed by the methods and systems herein, reflects an improvement in the functioning of distributed token systems.

### Blockchain Applications

Aspects disclosed herein utilize blockchains for building decentralized systems and applications. A blockchain is a data structure that guarantees the consistency of the data it contains. Data is stored in blocks and each block is linked together in a chain. Each block typically stores the cryptographic hash of the entire previous block, inclusive of the data and that block's hash of its previous block. Accordingly, any change or alteration to data in a block would require all subsequent block hashes need to be recomputed. Thus, a blockchain is able to track transactions and ensure data integrity.

The present disclosure takes advantage of the ability of blockchain to be used to store data. Cryptocurrencies use this data to store transactions. In some aspects, disclosed herein are platforms, systems, and methods for using blockchain to store data structures that comprise the hash of the data to be verified and the digital signature of that hash, which is created using the private key of the owner. In some cases, a plurality of these data structures is added to at least one block in the blockchain. Once added to the blockchain, that data is available for future verification. Implementations can choose to store a reference to the block which contains the hash, or the timestamp of when it was added to aid in future verifications.

Cryptocurrencies and similar technologies are based on blockchain technology. A blockchain is a record database that records every transaction and adds them to a growing list of records as blocks in the blockchain. In some embodiments, every computing node connected to the blockchain peer-to-peer (P2P) network maintains a full copy of the blockchain. Therefore, by analytically traversing the chain of transactions, a computing node can verify the data stored in individual transactions at any point in history such as, for example, a timestamp for a particular transaction. Any alteration to the transaction data would generate a contradictory hash from the block's hash stored in the succeeding block.

In some embodiments, a cryptographic transaction is a cryptographically signed message that transfers an amount of the cryptocurrency from a sender to a recipient address. In some embodiments, the transaction is encrypted using symmetric encryption or asymmetric encryption. In a preferred embodiment, the transaction is encrypted using asymmetric encryption. Asymmetric encryption utilizes private and public keypair for a given address. Thus, both a sender and a recipient will have a public and private keypair. The public key is used to encrypt a message or transaction, and the corresponding private key allows the encrypted transaction to be decrypted. The private key of such a keypair is used to sign transactions from the corresponding address of the sender. The signed transaction is then broadcasted to one or more nodes of the P2P network, and in some cases, every node of the cryptocurrency network. Because the public key is made available to the network, everyone is then able to verify the authenticity of the transaction using the public key from the sender's address.

Transactions can be organized into one or more blocks. A new block includes one or more transactions that have not yet been written into the existing blockchain. The blockchain has an initial or genesis block comprising data (e.g., details of a transaction) and a hash of the block's contents. Each subsequent block on the blockchain comprises data, a hash of the block's contents, and the hash of the previous block in the blockchain. In some embodiments, the data stored in the block comprises details of a transaction such as a sender, a recipient, transaction information (e.g., amount of a cryptocurrency transferred), and a timestamp. A newly created block is broadcasted and added to the growing blockchain. The new block is then validated by the P2P network through mechanisms such as proof of work or proof of stake. Blocks that are tampered with (e.g., the data is altered) will produce a different hash, which results in the block being rejected. Conversely, the validated block becomes part of the blockchain and is immutable, which means that the transactions of the block are now confirmed and cannot be changed or removed.

Every block in the blockchain contains a cryptographic hash of the previous block on the blockchain, which creates a chain from the first block (genesis block) to the current one. Because each block contains the cryptographic hash referencing the previous block and a hash of the contents of the current block, which is in turn referenced by the subsequent block, any modification of a single block will result in a different hash for every subsequent block on the chain. Thus, the P2P network is able to detect corruption or alteration of blocks in the blockchain all the way back to the most recent confirmed block. This process ensures the integrity of the chain.

For a distributed blockchain to function, the participants are generally required to agree on the current state of the blockchain. To achieve consensus, distributed blockchains usually require a timestamp and a “proof”. Two methods of establishing this proof are the “proof-of-work” algorithm and the “proof-of-stake” algorithm. The consensus algorithm produces a “proof” which is incorporated into the block and the subsequent block hash.

One non-limiting example of creating a block and appending it to the blockchain is called mining. Examples of mining algorithms include proof-of-work and proof-of-stake methods. The proof-of-work is a computationally intensive process that requires solving a unique and difficult math problem so that the number of blocks mined each day remains steady. Proof-of-work allows nodes on the network to reach a consensus on a block by having nodes on the network solve a cryptographic puzzle. In Bitcoin, all nodes (also called miners) in the network race to solve the cryptographic puzzle, and the first miner to find the solution gets a reward (e.g., an amount of Bitcoins). In some embodiments, the cryptographic puzzle is configured to require a great deal of trial and error in order to make it difficult to find a solution, while the solution is easy to verify. In Bitcoin, a proof-of-work scheme is SHA-256, which requires the SHA-256 hash of a block's header to be lower than or equal to a specific target value in order for the block to be valid. However, proof-of-work is computationally intensive and requires a significant amount of energy to power the miners. Proof-of-work also incentivizes server farms that benefit from economies of scale in purchasing and running application specific integrated circuit chips (ASICs) designed to conduct mining.

In some embodiments, a proof-of-stake method is used. Advantages of proof-of-stake include faster transaction validation and decreased power consumption. Proof-of-stake does not require all nodes in the P2P network to carry out the mining. Instead, a node (referred to as a validator) is chosen to validate the new block before it is added to the blockchain. Validators are selected or elected in part based on a “stake” the validator node deposits into the network. The stake can be an amount of a currency, a cryptocurrency, or other form of stake. The probability a validator has of being selected to validate the new block is based partly on the size of the stake. Thus, validators with larger stakes have a greater chance of being selected to validate a new block. In order to help ensure legitimacy of the validation process, proof-of-stake causes a validator to lose the stake if it approves fraudulent transactions. Generally, the stake is higher than the amount the validator can gain from transaction fees so as to incentivize validators to reject fraudulent transactions.

### Token-Split and Reverse-Split Applications

In some aspects, disclosed herein are methods for stabilizing the price of tokens in a cryptocurrency by splitting or reverse-splitting each token in the supply of tokens in an ecosystem, such as those disclosed herein. In some embodiments, a multiplication block is integrated into the distributed ledger computer network, which multiplies the supply of tokens by a number in a token ecosystem. In some embodiments, the supply of tokens is multiplied by a number that is greater than one, resulting in one token in the supply now representing more than one token, thereby expanding of the supply of tokens. In another embodiment, the supply of tokens is multiplied by a number that is lower than one, resulting in one token in the supply now representing less than one token, thereby contracting of the supply of tokens. Non-limiting examples of the number comprise whole numbers, fractions, and decimals.

There are several advantages of the present methods. The token-split and reverse-split methods disclosed herein prevent the influx and efflux of tokens in an ecosystem, thereby reducing the need for complex algorithms to calculate the number of tokens to add or subtract from the ecosystem. In addition, the token-split and reverse-split methods reduce the volatility of token price due to the pressures of high demand that serves to impede the ability of stakeholders to make future investment decisions efficiently. The present methods, and systems, networks, and media that employ them, provide significant improvements to the historical means for managing a supplies of currencies using a scarcity model, wherein high demand combined with diminishing supply yields significant appreciation of token price, making it nearly impossible to ensure efficient decision-making regarding future transactions.

### Central Authority

In aspects, disclosed herein, a central authority is granted the right to manage the supply of tokens in a token ecosystem, such as the utility token ecosystem disclosed herein. The central authority is granted authority to insert the multiplication block into the distributed ledger computer network. In some embodiments, the multiplication block is inserted to the distributed ledger computer network using a special purpose central authority private key that is compatible with a public key. Such an approach would be best where there are no independent, external sources that could serve as a good guide for determining the optimal amount of tokens that should be in circulating in the ecosystem to maximize its efficiency (e.g., where the decision requires the weighing of a variety of complex factors that are difficult to predict into the future).

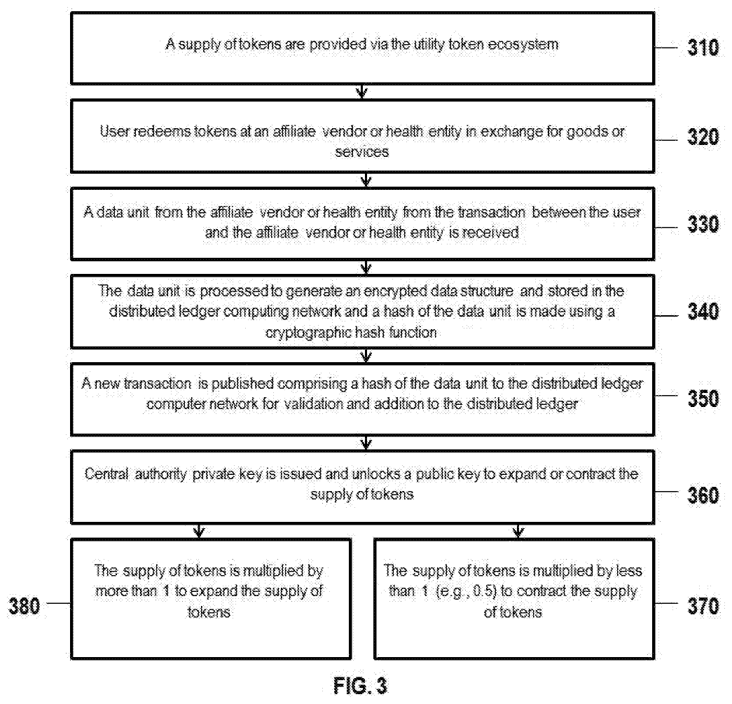

The token-split and reverse-split methods disclosed herein may be used in the personal health tracking system and utility token network disclosed herein. As shown in FIG. 3, a supply of tokens is provided to the utility token ecosystem 310, and the user acquires a token from a health entity 150, an affiliate vendor 120, or the utility token management system 140 as disclosed herein. Next, the user redeems the tokens at an affiliate vendor or health entity in exchange for a good, a service, or both 320. In some embodiments, the token is further recalled from the user, by the affiliate vendor, the heath entity, or any combination thereof.

A data unit from the affiliate vendor or health entity from the transaction between the user and the affiliate vendor or the health entity is then received 330. The data unit is processed to generate an encryption data structure and stored in the distributed ledger computing network and a hash of the data is made using a cryptographic hash function 340. Next, a new transaction is published comprising a hash of the data unit to the distributed ledger computer network for validation and addition to the distributed ledger 350. A central authority private key is issued and unlocks a public key to expand or contract the supply of tokens 360. The supply of tokens is multiplied by a number using a multiplication block inserted into the distributed ledger computer network by the central authority. In some embodiments, the supply of tokens is multiplied by a number that is greater than one, resulting in one token in the supply now representing more than one token, thereby expanding of the supply of tokens 380. In another embodiment, the supply of tokens is multiplied by a number that is lower than one, resulting in one token in the supply now representing less than one token, thereby contraction of the supply of tokens 370.

### Off-Chain Oracle

In aspects disclosed herein, an off-chain oracle is integrated into the distributed ledger computer network and acts as the multiplication block disclosed herein by multiplying the supply of tokens by a number that is greater or less than one, if the price of a token in the supply of tokens deviates from a reference price. In some embodiments, the reference price is predetermined. In some embodiments, the reference price is based on external stimulus. In some embodiments, the external stimulus maintains a relatively static value. Non-limiting examples of external stimuli include price of a commodity, or the value of an established currency, such as the U.S. dollar or the Euro. In some embodiments, the price of a token in the supply of tokens deviates from the reference price by plus or minus 1%, 2%, 3%, 4%, 5%, 6%, 7%, 8%, 9% or 10% of the reference price. In some embodiments, the price a token in the supply of tokens deviates from the reference price if the price of a token is below or exceeds the reference price by any amount.

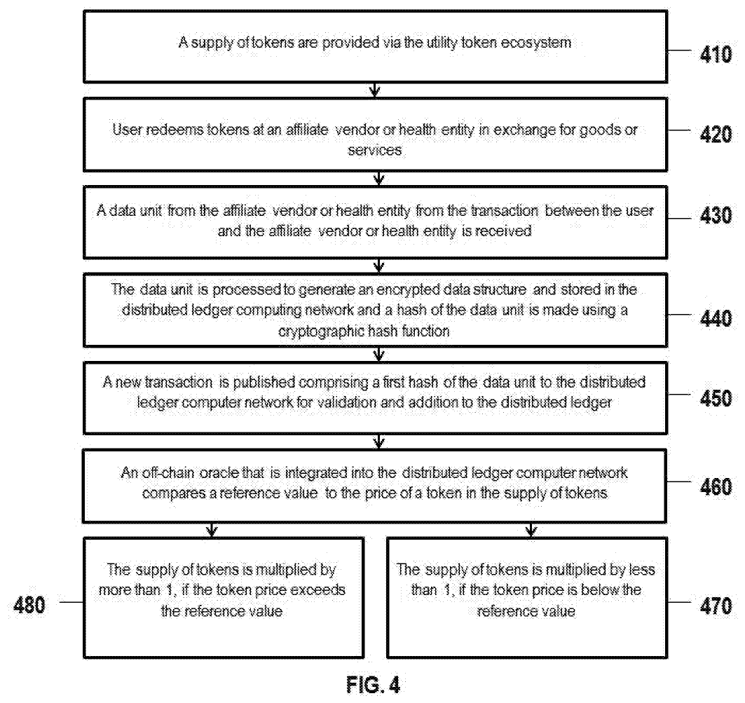

The token-split and reverse-split methods disclosed herein may be used in the personal health tracking system and utility token network disclosed herein. As shown in FIG. 4, a supply of tokens is provided to the utility token ecosystem 410, and the user acquires a token from a health entity 150, an affiliate vendor 120, or the utility token management system 140 as disclosed herein. Next, the user redeems the tokens at an affiliate vendor or health entity in exchange for a good, a service, or both 420. In some embodiments, the token is further recalled from the user, by the affiliate vendor, the heath entity, or any combination thereof.

A data unit from the affiliate vendor or health entity from the transaction between the user and the affiliate vendor or the health entity is received 430. The data unit is processed to generate an encryption data structure and stored in the distributed ledger computing network and a hash of the data is made using a cryptographic hash function 440. Next, a new transaction is published comprising a hash of the data unit to the distributed ledger computer network for validation and addition to the distributed ledger 450. An off-chain oracle that is integrated into the distributed ledger computer network compares the reference price to the price of a token in the supply of tokens 460. The off-chain oracle multiplies the supply of tokens is multiplied by a number. In some embodiments, the supply of tokens is multiplied by a number that is greater than one, resulting in one token in the supply now representing more than one token, thereby expanding of the supply of tokens 480. In another embodiment, the supply of tokens is multiplied by a number that is lower than one, resulting in one token in the supply now representing less than one token, thereby contraction of the supply of tokens 470.

### Health Tracker Blockchain Applications

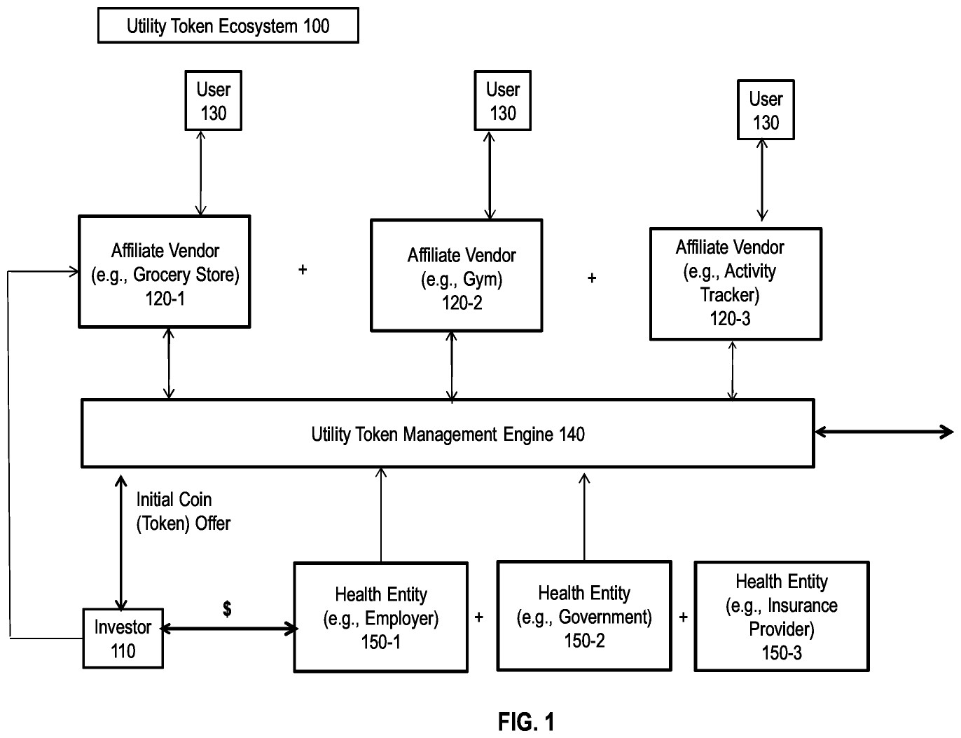

In some aspects, disclosed herein are ecosystems, systems, and methods for tracking user compliance with a health program, by tracking one or more data structures stored on a blockchain. One embodiment of the utility token ecosystem 100 is illustrated in FIG. 1. In some embodiments, a supply of tokens is sold to an investor 110, or group of investors, in exchange for currency. In some embodiments, the currency comprises the United States dollar. In other embodiments, the currency comprises cryptocurrencies such as those disclosed in the present application. The investor sells the tokens to one or more health entities 150-1, 150-2, 150-3, in exchange for currency. The one or more health entities 150-1, 150-2, 150-3 can further provide the tokens to the utility token management engine 140. A health entity is an entity that has an interest in promoting the health of its constituents. Non-limiting examples of a health entity 150 include a health care provider, government, or insurance provider. The health entity 150 purchases tokens from the investor as a means to reward constituents of the health entity, like a user 130, for taking steps to meet one or more health-related goals, such as engaging in transactions to improve user health or engaging in healthy behavior. In addition, or alternatively, the investor sells tokens to an affiliate vendor 120, in exchange for currency. An affiliate vendor 120 purchases tokens from the investor as a means to reward constituents of the affiliate vendor 120, like a user 130 (e.g., customer or client), for taking steps to meet one or more health-related goals, such as engaging in transaction to improve user health, or engaging in healthy behavior. Non-limiting examples of health-related goals comprise goals to lose weight, lower blood pressure, maintain glucose levels, and lower stress. Non-limiting examples of transactions include purchasing healthy food at a grocery store that is an affiliate vendor 120-1, attending a fitness class, or a yoga class, at a gym that is an affiliate vendor 120-2, or exercising using a fitness tracker 120-3. Non-limiting examples of healthy behavior include any activity to improve health, such as eating a healthy meal, losing weight, experiencing few migraines, completion of an athletic event or training milestone, completion of a diet milestone and the like.

In some embodiments, the affiliate vendor 120, health entity 150, or both, lends tokens to the utility token management engine 140 to store until a transaction to improve user health is completed by the user 130. In some embodiments, data units comprising transactions to improve user health between the user 130 and the health entity 150 or the affiliated vendor 120 are fed through a hashing algorithm to generate a hash of the data. This hash can be stored on a block along with the address or public key and the digital signature. Multiple such data structures or transactions containing the data hash, address, and signature can be stored in the blocks of the blockchain along with the hash of the previous block. In some embodiments, the utility token management engine 140 receives a data unit from an affiliate vendor 120 comprising a transaction to improve user health between the user 130 and the affiliate vendor 120. In some embodiments, the utility token management engine 140 notifies the health entity 150 of the user's 130 transaction history verifying that the user 130 engaged in the transaction and is a constituent of the health entity 150. In some embodiments the utility token management engine 140 provides a report with a transaction summary. In response to the notification, the health entity 150 rewards the user 130 for taking steps to meeting one or more health related goals by giving the user 130 one or more tokens. The tokens may be redeemed by the user 130 at an affiliate vendor 120, health entity 150, or sold to a third party. In some embodiments, the utility token management engine 140 sells the tokens to third parties, or other entities with an interest in promoting health. In some embodiments, the token can be recalled from the user, by the affiliate vendor, the heath entity, or any combination thereof. In some embodiments, a token created through a split in the tokens can be recalled from the user, by the affiliate vendor, the heath entity, or any combination thereof. Recalling tokens can enable the issuer of tokens to maintain a degree of token liquidity. For example, demand for tokens from new health insurer can be addressed by the creation of more tokens through a token split. However, unless current investors (speculators) subsequently sell the additional tokens to the new health insurer (directly or indirectly), the tokens remain illiquid. If the tokens were traded on an exchange, additional tokens can be created until there is sufficient token supply at a target token price. However, as, in some cases, the token trading herein do not employ a token exchange, a token recall is supplemented to maintain said liquidity.

In some embodiments, the transaction from the utility token management engine 140 to the investor 110 is an initial coin or token offer. In some embodiments, the transaction from the investor 110 to the utility token management engine 140 is a cyber currency. In some embodiments, the transaction from the user 130 to the first affiliate vendor 120-1 is a notification that a food that is healthy to the user 130 has been consumed by the user 130. In some embodiments, the transaction from the first affiliate vendor 120-1 to the user 130 is a premium health food, a discount, or both.

In some embodiments, the transaction from the user 130 to the second affiliate vendor 120-2 is a notification that the user 130 has performed a healthy activity (e.g., attending a CrossFit class). In some embodiments, the transaction from the second affiliate vendor 120-2 to the user 130 is a physical exercise discount (e.g., a gym coupon).

In some embodiments, the transaction from the user 130 to the third affiliate vendor 120-3 is a notification that the user 130 has performed a healthy activity (e.g., jogging 4 km) via, for example, an activity tracker. In some embodiments, the transaction from the third affiliate vendor 120-3 to the user 130 is a health benefit (e.g., a hospital discount).

### Blockchain Platforms

Disclosed herein, in various aspects, are systems, methods, media, and devices for tracking user compliance with a health program using one or more blockchain platforms. Non-limiting examples of blockchain platforms or protocols include Bitcoin, Ethereum, Cardano, MultiChain, Zcash, Litecoin, Namecoin, Swiftcoin, Bytecoin, Peercoin, Emercoin, Feathercoin, Gridcoin, Primecoin, Ripple, Auroracoin, Dash, Zetacoin, Monero, Tether, Mazacoin, Vertcoin, and Zcash. In some embodiments, Ethereum is used as the blockchain platform. Ethereum is a public, open-source, blockchain-based decentralized computing protocol that features smart contracts functionality (e.g., scripting). The protocol provides a decentralized virtual machine called the Ethereum Virtual Machine (EVM), which carries out Turning-complete scripts by using a network of nodes and a token called ether or gas. The ether or gas is used to protect from network spam and to ensure proportional allocation of resources based on the incentive provided by any given request. Ethereum provides the ability to use smart contracts that can self-execute upon the satisfaction of certain conditions built into the contract. In some embodiments, a block containing one or more smart contracts is generated and published to the blockchain.

### Decentralized Blockchain Network

Disclosed herein, in various aspects, are systems, methods, media, and devices that utilize blockchain technology implemented on a decentralized peer-to-peer blockchain network. In some embodiments, the network comprises a plurality of nodes. In some embodiments, the plurality of nodes is not controlled by a single entity. In some embodiments, less than 51% of the plurality of nodes is controlled by a single entity. In some embodiments, no single entity or cartel of entities controls more than 1%, 5%, 10%, 15%, 20%, 30%, 40%, 50%, 51%, 60%, 70%, 80%, or 90% of the nodes in the network. In some embodiments, the network comprises nodes that are selected to prevent a 51% attack on the network. In some embodiments, the network is a public blockchain network that allows any participate to join the network as a node in executing the consensus protocol and maintaining the public, shared ledger.

In some embodiments, the network is a private blockchain network that is not open to everyone and may require permission or invitation for a participant to join the network. In some embodiments, a private blockchain network is controlled by a single entity. A private blockchain network can have advantages over public blockchain networks in that the transactions stored in the blocks has greater privacy since the blockchain is not publicly available. In addition, a private blockchain network controlled by a single entity would be responsible for writing and verifying each block. However, such a network would not offer the decentralized security of a public network. In such embodiments, a private blockchain network allows the blockchain to act as an efficient and private ledger or data store.

In some embodiments, a blockchain network is a consortium blockchain network that comprises a non-public group of trusted nodes. In some embodiments, a blockchain network such as a consortium blockchain network comprises nodes belonging to or controlled by known entities. In some embodiments, the known entities have an interest in promoting public health, or health of its constituents. In some embodiments, the entities include government entities (e.g., FDA, NIH), nonprofit organizations, private enterprises, health enterprises or organizations (e.g., hospitals), educational institutions (e.g., universities), scientists or research teams, and other organizations. As an example, in some embodiments, a pre-selected group of entities collaborate in a private blockchain network and participate in adding transactions to the growing blockchain as they collectively engage in a collaborative research project. In this example, a research team at a university engages in basic research on a new drug compound and obtains animal study data showing efficacy in disease treatment. Successful validation of this data on an Ethereum blockchain by the private network triggers a smart contract on the blockchain that releases investor funds (in the form of a cryptocurrency) in accordance with the established milestone built into the smart contract. The funds are released to a private company collaborating with the research group to finance phase I clinical trials.

In some embodiments, the nodes in the consortium network are controlled by trusted entities such that no significant percentage of the network is controlled by one entity or a group of entities that are allied or have an alignment of interests. In some embodiments, a consortium blockchain network provides limited permission to write new blocks to the blockchain (e.g., not all nodes can generate new blocks). Thus, consortium blockchain networks provide a hybrid between the increased efficiency and privacy of a private blockchain network with the decentralized security of a public blockchain network.

### Personal Health Tracking System

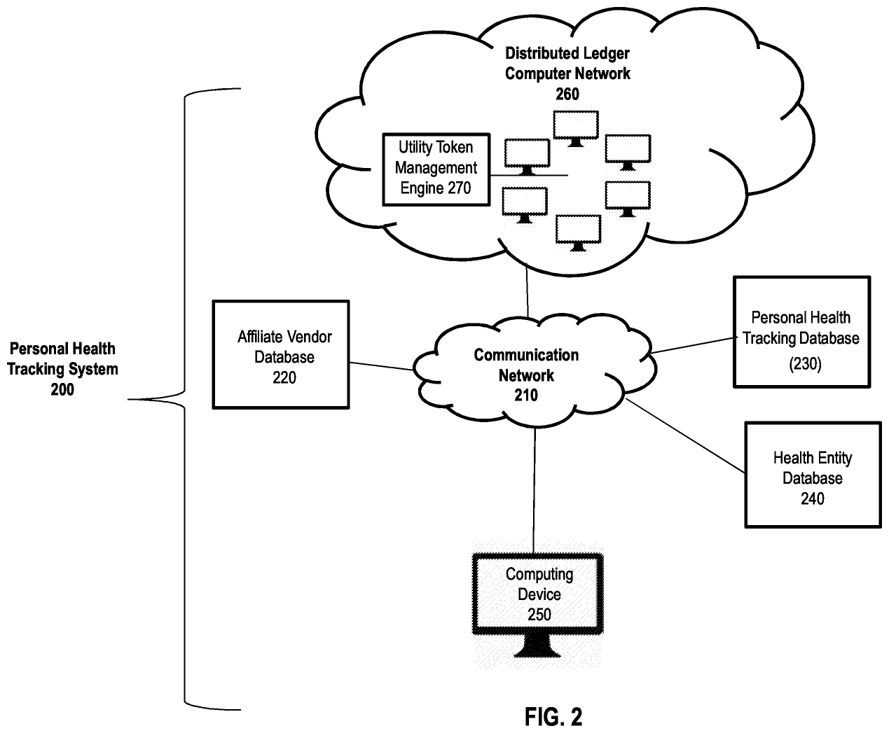

Disclosed herein, in various aspects, is a personal health tracking system as shown in FIG. 2. In some embodiments, the personal health tracking system comprises a network interface configured to communicate via a communication network 210, a computing device 250 in communication with a plurality of computing devices via a distributed ledger computing network 260. The computing device comprises at least one processor, a memory, and a computer program with instructions executable by the processor to track user compliance with a health program. In some embodiments, the processor of the computing device 250 executes instructions for storing, in a personal health database 230 a profile associated with an account belonging to a user. In some embodiments, instructions provide for storing, in a health entity database 240 a profile associated with an account belonging to the health entity. In yet other embodiments, instructions provide for storing, in an affiliate vendor database 220 a profile associated with an account belonging to an affiliate vendor. In some embodiments, the profiles disclosed herein comprise health-related goals for the user.

In an aspect disclosed herein, the user's compliance with the health-related goals determined by the user, the health entity, the affiliated vendor, or a combination thereof, is tracked using the utility token ecosystem shown in FIG. 1, on the distributed ledger computer network 260.

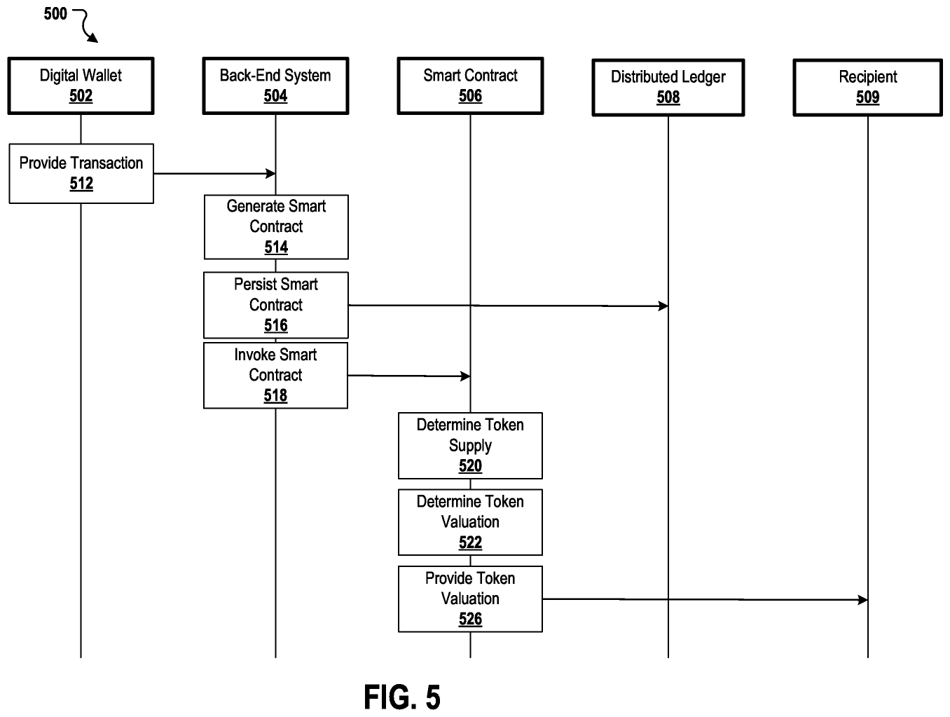

FIG. 5 depicts a flowchart of an example process 500. The example process 500 can be implemented by the various elements of the described utility token ecosystem. As depicted, the example process shows in more detail that communication as well as the separation of work between a digital wallet 502, a back-end system 504, a smart contract 506, a distributed ledger 508, and a recipient 509. The flowcharts generally show how a transaction is generated, processed, and persisted to the distributed ledger 508. For clarity of presentation, the description that follows generally describes the example process 500 in the context of FIGS. 1-4. However, it will be understood that the process 500 may be performed, for example, by any other suitable system, environment, software, and hardware, or a combination of systems, environments, software, and hardware as appropriate. In some embodiments, various operations of the process 500 can be run in parallel, in combination, in loops, or in any order.

At 512, the first transaction is provided by the digital wallet 502 to the back-end system 504. In some embodiments, the digital wallet 502 is associated with a key address. In some embodiments, the first transaction includes a recipient and a token, from a plurality of tokens, associated with the key address. In some embodiments, the system includes a second digital wallet 502 associated with a second key address. In some embodiments, the digital wallet 502 provides a transfer transaction to the back-end system 504. In some embodiments, the transfer transaction includes the token and the second key address. In some embodiments, the back-end system 504, when receiving the transfer transaction from the digital wallet 502, generates a transfer block that includes the transfer transaction and persists the transfer block persists to the distributed ledger 508. In some embodiments, the utility token system includes a peer-to-peer network. In some embodiments, the peer-to-peer network includes a plurality of nodes. In some embodiments, the transfer block is verified by the nodes. From 512, the process 500 proceeds to 514.

At 514, the back-end system 504 generates the smart contract 506 associated with the tokens. In some embodiments, the smart contract 506 includes a set of instructions that are executed by one or more processors. From 514, the process 500 proceeds to 516.

At 516, the back-end system 504 persists the smart contract 506 to the distributed ledger 508. In some embodiments, the back-end system 504 receives, from an affiliate vendor, a second transaction associating the token with the key address, generates a block comprising the second transaction, and persists the block to the distributed ledger 508. From 516, the process 500 proceeds to 518.

At 518, the back-end system 504 invokes the smart contract 506 with the first transaction when receiving the first transaction from the digital wallet 502. From 518, the process 500 proceeds to 520.

At 520, the smart contract 506 is executed to determine a current supply of the tokens based on a multiplication block stored to the distributed ledger 508. In some embodiments, the token represents a portion of the current supply of the tokens. In some embodiments, the back-end system 504 receives the multiplication block signed by a key associated with a central authority, verifies the signature based on a public address of the key, and persists the multiplication block to the distributed ledger 508. In some embodiments, the utility token system includes a peer-to-peer network. In some embodiments, the peer-to-peer network includes a plurality of nodes. In some embodiments, the multiplication block is verified by a plurality of the nodes. In some embodiments, persisting the multiplication block generates a hard fork of the distributed ledger 508. In some embodiments, the current supply of the tokens is determined based on an off-chain oracle instead of the multiplication block. In some embodiments, the multiplication block comprises a multiplier for the number of tokens. In some embodiments, the multiplier is determined based on a reference price for each of the tokens. In some embodiments, the reference price is predetermined. In some embodiments, the reference price comprises an initial price for the tokens. In some embodiments, the reference price mirrors a price of an existing currency or cryptocurrency. In some embodiments, the multiplier comprises a decimal value or a fractional value. In some embodiments, the recipient 509 comprises an affiliate vendor, and the valuation of the token determines a discount, rebate, or coupon for the affiliate vendor. From 520, the process 500 proceeds to 522.

At 522, the smart contract 506 is executed to determine a valuation of the token based on the current supply of the tokens. From 522, the process 500 proceeds to 524.

At 524, the smart contract 506 is executed to provide the valuation of the token to the recipient 509. From 524, the process 500 ends.

### Computing System

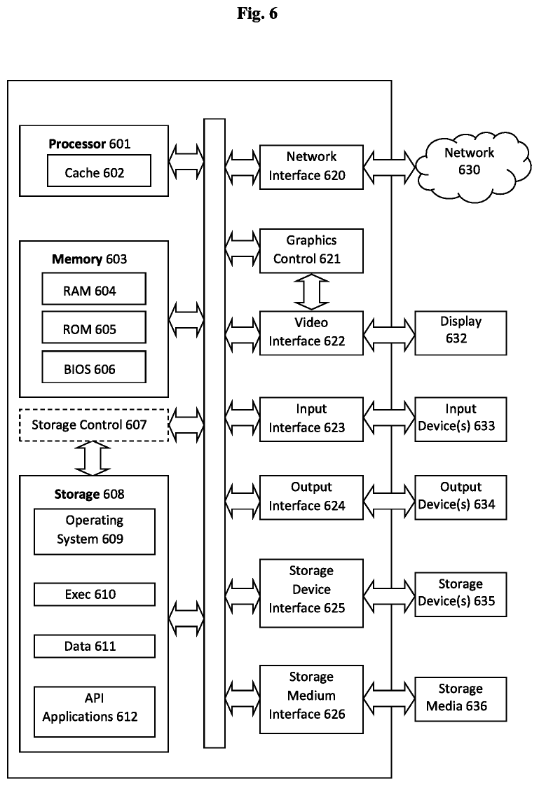

Referring to FIG. 6, a block diagram is shown depicting an exemplary machine that includes a computer system 600 (e.g., a processing or computing system) within which a set of instructions can execute for causing a device to perform or execute any one or more of the aspects and/or methodologies for static code scheduling of the present disclosure. The components in FIG. 6 are examples only and do not limit the scope of use or functionality of any hardware, software, embedded logic component, or a combination of two or more such components implementing particular embodiments.

Computer system 600 may include one or more processors 601, a memory 603, and a storage 608 that communicate with each other, and with other components, via a bus 640. The bus 640 may also link a display 632, one or more input devices 633 (which may, for example, include a keypad, a keyboard, a mouse, a stylus, etc.), one or more output devices 634, one or more storage devices 635, and various tangible storage media 636. All of these elements may interface directly or via one or more interfaces or adaptors to the bus 640. For instance, the various tangible storage media 636 can interface with the bus 640 via storage medium interface 626. Computer system 600 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 600 includes one or more processor(s) 601 (e.g., central processing units (CPUs) or general purpose graphics processing units (GPGPUs)) that carry out functions. Processor(s) 601 optionally contains a cache memory unit 602 for temporary local storage of instructions, data, or computer addresses. Processor(s) 601 are configured to assist in execution of computer readable instructions. Computer system 600 may provide functionality for the components depicted in FIG. 6 as a result of the processor(s) 601 executing non-transitory, processor-executable instructions embodied in one or more tangible computer-readable storage media, such as memory 603, storage 608, storage devices 635, and/or storage medium 636. The computer-readable media may store software that implements particular embodiments, and processor(s) 601 may execute the software. Memory 603 may read the software from one or more other computer-readable media (such as mass storage device(s) 635, 636) or from one or more other sources through a suitable interface, such as network interface 620. The software may cause processor(s) 601 to carry out one or more processes or one or more steps of one or more processes described or illustrated herein. Carrying out such processes or steps may include defining data structures stored in memory 603 and modifying the data structures as directed by the software.

The memory 603 may include various components (e.g., machine readable media) including, but not limited to, a random access memory component (e.g., RAM 604) (e.g., static RAM (SRAM), dynamic RAM (DRAM), ferroelectric random access memory (FRAM), phase-change random access memory (PRAM), etc.), a read-only memory component (e.g., ROM 605), and any combinations thereof. ROM 605 may act to communicate data and instructions unidirectionally to processor(s) 601, and RAM 604 may act to communicate data and instructions bidirectionally with processor(s) 601. ROM 605 and RAM 604 may include any suitable tangible computer-readable media described below. In one example, a basic input/output system 606 (BIOS), including basic routines that help to transfer information between elements within computer system 600, such as during start-up, may be stored in the memory 603.

Fixed storage 608 is connected bidirectionally to processor(s) 601, optionally through storage control unit 607. Fixed storage 608 provides additional data storage capacity and may also include any suitable tangible computer-readable media described herein. Storage 608 may be used to store operating system 609, executable(s) 610, data 611, applications 612 (application programs), and the like. Storage 608 can also include an optical disk drive, a solid-state memory device (e.g., flash-based systems), or a combination of any of the above. Information in storage 608 may, in appropriate cases, be incorporated as virtual memory in memory 603.

In one example, storage device(s) 635 may be removably interfaced with computer system 600 (e.g., via an external port connector (not shown)) via a storage device interface 625. Particularly, storage device(s) 635 and an associated machine-readable medium may provide non-volatile and/or volatile storage of machine-readable instructions, data structures, program modules, and/or other data for the computer system 600. In one example, software may reside, completely or partially, within a machine-readable medium on storage device(s) 635. In another example, software may reside, completely or partially, within processor(s) 601.

Bus 640 connects a wide variety of subsystems. Herein, reference to a bus may encompass one or more digital signal lines serving a common function, where appropriate. Bus 640 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 600 may also include an input device 633. In one example, a user of computer system 600 may enter commands and/or other information into computer system 600 via input device(s) 633. Examples of an input device(s) 633 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 633 may be interfaced to bus 640 via any of a variety of input interfaces 623 (e.g., input interface 623) including, but not limited to, serial, parallel, game port, USB, FIREWIRE, THUNDERBOLT, or any combination of the above.

In particular embodiments, when computer system 600 is connected to network 630, computer system 600 may communicate with other devices, specifically mobile devices and enterprise systems, distributed computing systems, cloud storage systems, cloud computing systems, and the like, connected to network 630. Communications to and from computer system 600 may be sent through network interface 620. For example, network interface 620 may receive incoming communications (such as requests or responses from other devices) in the form of one or more packets (such as Internet Protocol (IP) packets) from network 630, and computer system 600 may store the incoming communications in memory 603 for processing. Computer system 600 may similarly store outgoing communications (such as requests or responses to other devices) in the form of one or more packets in memory 603 and communicated to network 630 from network interface 620. Processor(s) 601 may access these communication packets stored in memory 603 for processing.

Examples of the network interface 620 include, but are not limited to, a network interface card, a modem, and any combination thereof. Examples of a network 630 or network segment 630 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 630, may employ a wired and/or a wireless mode of communication. In general, any network topology may be used.

Information and data can be displayed through a display 632. Examples of a display 632 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 632 can interface to the processor(s) 601, memory 603, and fixed storage 608, as well as other devices, such as input device(s) 633, via the bus 640. The display 632 is linked to the bus 640 via a video interface 622, and transport of data between the display 632 and the bus 640 can be controlled via the graphics control 621. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 632, computer system 600 may include one or more other peripheral output devices 634 including, but not limited to, an audio speaker, a printer, a storage device, and any combinations thereof. Such peripheral output devices may be connected to the bus 640 via an output interface 624. Examples of an output interface 624 include, but are not limited to, a serial port, a parallel connection, a USB port, a FIREWIRE port, a THUNDERBOLT port, and any combinations thereof.

In addition or as an alternative, computer system 600 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

### Computing Device

In various embodiments, the subject matter described herein include a computing device, or use of the same. In further embodiments, the computing device includes one or more hardware central processing units (CPU) that carry out the device's functions. In still further embodiments, the computing device further comprises an operating system configured to perform executable instructions. In some embodiments, the computing device is optionally connected a computer network. In further embodiments, the computing device is optionally connected to the Internet such that it accesses the World Wide Web. In still further embodiments, the computing device is optionally connected to a cloud computing infrastructure. In other embodiments, the computing device is optionally connected to an intranet. In other embodiments, the computing device is optionally connected to a data storage device.

In accordance with the description herein, suitable computing devices include, by way of non-limiting examples, server computers, desktop computers, laptop computers, notebook computers, sub-notebook computers, netbook computers, netpad computers, set-top computers, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the computing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the computing device includes a display to send visual information to a user. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In still further embodiments, the display is a combination of devices such as those disclosed herein.

In some embodiments, the computing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera to capture motion or visual input. In still further embodiments, the input device is a combination of devices such as those disclosed herein.

### Non-Transitory Computer Readable Storage Medium

In various embodiments, the subject matter disclosed herein include one or more non-transitory computer readable storage media encoded with a program including instructions executable by the operating system. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In various embodiments, the subject matter disclosed herein include at least one computer program, or use of the same. A computer program includes a sequence of instructions, executable in the computing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages. The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Software Modules

In various embodiments, the subject matter disclosed herein include at least one software module, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In various embodiments, the subject matter disclosed herein include at least one database, or use of the same. In view of the disclosure provided herein, those of skill in the art will recognize that many databases are suitable for storage and retrieval of blockchain, transaction, domain name, routing, and virtual blockchain information. In various embodiments, suitable databases include, by way of non-limiting examples, relational databases, non-relational databases, object oriented databases, object databases, entity-relationship model databases, associative databases, and XML databases. Further non-limiting examples include LevelDB, SQL, SQLite, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices. In some embodiments, a database is based on one or more blockchains published on a distributed/decentralized network.

### Non-Transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable by one or more processor(s) of the computing device's CPU, written to perform a specified task. Computer readable instructions may be implemented as program modules, such as functions, objects, Application Programming Interfaces (APIs), computing data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™ and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® ActionScript, JavaScript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

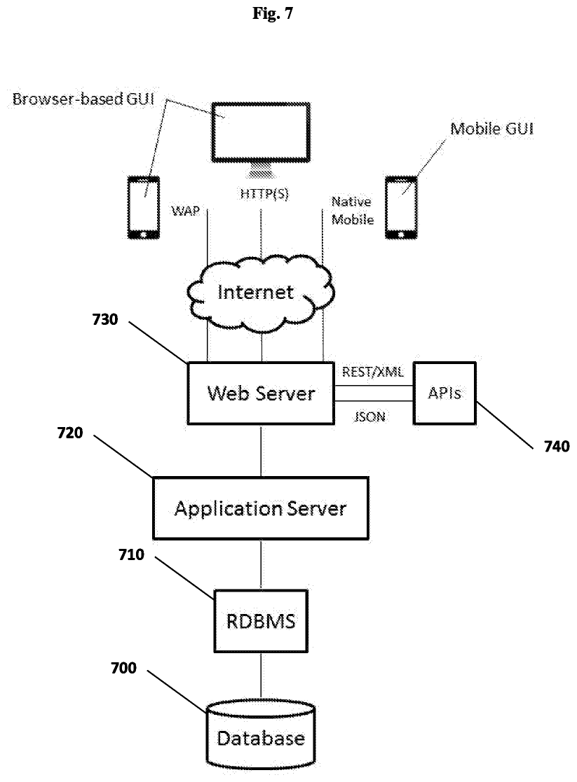

Referring to FIG. 7, in a particular embodiment, an application provision system comprises one or more databases 700 accessed by a relational database management system (RDBMS) 710. Suitable RDBMSs include Firebird, MySQL, PostgreSQL, SQLite, Oracle Database, Microsoft SQL Server, IBM DB2, IBM Informix, SAP Sybase, SAP Sybase, Teradata, and the like. In this embodiment, the application provision system further comprises one or more application severs 720 (such as Java servers, .NET servers, PHP servers, and the like) and one or more web servers 730 (such as Apache, IIS, GWS and the like). The web server(s) optionally expose one or more web services via app application programming interfaces (APIs) 740. Via a network, such as the Internet, the system provides browser-based and/or mobile native user interfaces.

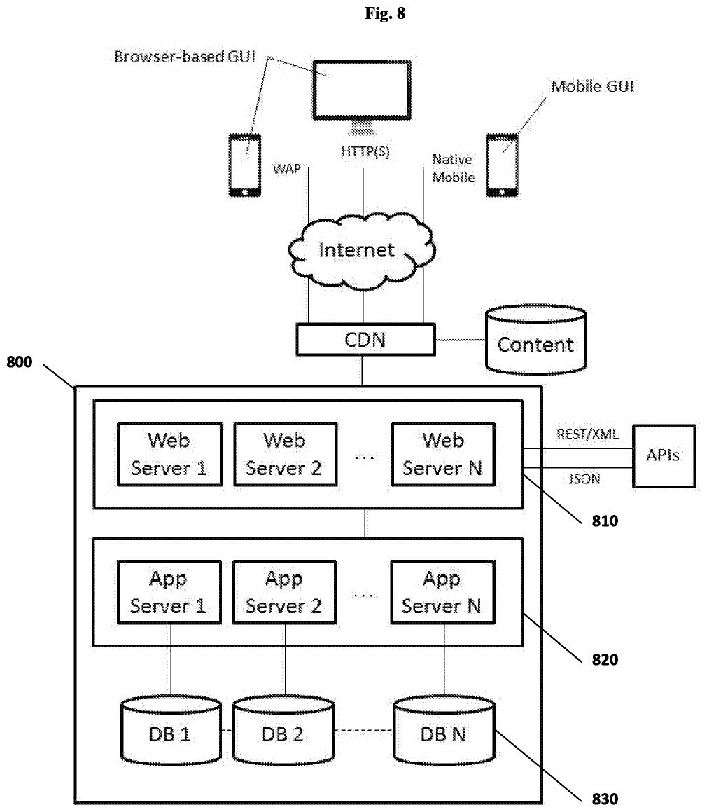

Referring to FIG. 8, in a particular embodiment, an application provision system alternatively has a distributed, cloud-based architecture 800 and comprises elastically load balanced, auto-scaling web server resources 810 and application server resources 820 as well synchronously replicated databases 830.

### Mobile Application

In some embodiments, a computer program includes a mobile application provided to a mobile computing device. In some embodiments, the mobile application is provided to a mobile computing device at the time it is manufactured. In other embodiments, the mobile application is provided to a mobile computing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome Web Store, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Web Browser Plug-in

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET, or combinations thereof.

Web browsers (also called Internet browsers) are software applications, designed for use with network-connected computing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software Opera Mobile, and Sony PSP™ browser.

### Terms and Definitions

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” refers to an amount that is near the stated amount by 10%, 5%, or 1%, including increments therein.

As used herein, the term “about” in reference to a percentage refers to an amount that is greater or less the stated percentage by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, a “hash function” or “hashing algorithm” refers to any function that can transform input data of an arbitrary size into a string or hash of a fixed size. In some embodiments, a hash function or algorithm is a cryptographic hash function which is a one-way function, thus making it extremely difficult to determine the input based on the hash output. The cryptographic hash function is configured to always produce the same hash when given the same input. Accordingly, any alteration to the input data can be detected by comparing the hash of the current input data to the hash of the earlier input data. If the data has remained unchanged, the hashes will be identical. Examples of cryptographic hash functions include UMAC, VMAC, PMAC, HMAC, MD6, BLAKE2, BLAKE-256, BLAKE-512, BLAKE2s, BLAKE2b, ECOH, FSB, GOST, HAS-160, HAVAL, MD2/4/5/6, RIPEMD-128/160/320, and SHA-1/3/224/256/384/512.

As used herein, a “blockchain” refers to a list of records (also referred to as blocks), which are cryptographically linked and secured. The blockchain is typically continuously growing as new blocks are added to the chain. Each block may contain a cryptographic hash of the previous block, a timestamp (e.g., date/time), and one or more transactions. In some cases, a “blockchain,” as described herein, is implemented via a decentralized computing network to form a distributed database or distributed ledger. The decentralized computing network can be a public or private network. A public blockchain network is open to the public such that any participate can join the network as a node in executing the consensus protocol and maintaining the public, shared ledger. A private blockchain network is not open to everyone and may require permission or invitation for a participant to join the network.

As used herein, a “data unit” refers to any amount of electronic data or information. Examples of data units include electronic files, communications, documents, images, videos, contracts, other forms of data, and components thereof. In some cases, a single electronic file (e.g., a document, email, image, etc) is composed of multiple component data units. Thus, a block in a blockchain may contain one or more data units that make up a portion of one or more electronic files.

As used herein, “verification” refers to confirmation that the data or data unit being verified is unaltered. For example, a data unit can be verified by comparing its hash value to the hash value of the original data unit that was stored on a blockchain. Identical hash values would indicate that the data unit being verified is unaltered from the original data unit on the blockchain.

As used herein, “certification” refers to providing a certificate or other formal signifier of the integrity of a data or data unit (e.g., an electronic health record). For example, once a data unit has been verified, a certificate formally attesting to the accuracy and integrity of the data unit may be issued and delivered to a user and/or third party requesting certification.

As used herein, “token,” refers to a unit of value in a distributed ledger computer network that is useful for receiving or making payments on the distributed ledger computer network. In some embodiments, a token refers to a form of cryptocurrency. As used herein, the term “coin” is used interchangeably with the term, “token.”

As used herein, “token-split,” refers to the process of transforming a token in a supply tokens in an ecosystem into more than one token. In some embodiments, the entire supply of tokens is subject to a token-split.

As used herein, “reverse-split,” refers to the process of transforming a token in a supply of tokens in an ecosystem into less than one token. In some embodiments, the entire supply of tokens is subject to a reverse-split. As used herein, the term “reverse-split” is used interchangeably with the term, “amalgamation.”

As used herein, “smart contracts,” refer to digitization of the legal contracts and include executable code which represents, for example, contract terms. As such, a smart contract not only defines the rules and penalties related to an agreement in the same way that a traditional contract does, but also automatically enforces those obligations. A smart contract may accomplish this by taking information as input, assigning a value to that input through the rules set out in the contract, and executing the actions required by those contractual clauses. For example, a smart contract may determine whether an asset should be sent to a destination entity or whether it should be returned to an originating entity. Smart contracts may be coded in a programming language, such as Solidity™ For example, a smart contract may be programed to deliver payment when an item is received. In this format, a contract is converted to computer code, stored and replicated on the system, and supervised by a network of computers that run the blockchain. Smart contracts can call other smart contract just like an Object-oriented object to create and use objects of another class. Smart contracts can store data. The data stored can be used to record information, fact, associations, balances and any other information needed to implement logic for real world contracts. In some embodiments, a smart contract is deployed, stored, and executed within the virtual machine.

As used herein, “digital wallet,” refers to a device, physical medium, program or a service that stores public and/or private keys and interacts with various distributed ledger (e.g., blockchain) to enable users to send and receive digital currency and monitor their balance. For example, in some embodiments, digital wallet can be used to track ownership, receive digital wallet, or spend cryptocurrencies. In some embodiments, the cryptocurrency itself is not in the wallet. For example, in case of bitcoin and cryptocurrencies derived from it, the cryptocurrency is decentrally stored and maintained in a publicly available ledger called the blockchain. There are several types of wallets that provide different ways to store and access digital currency. For example, digital wallets can be broken down into three distinct categories—software, hardware, and paper. Software wallets can further be broken down into desktop, mobile or online wallets. In general, desktop wallets are downloaded and installed on a PC or laptop, online wallets run on the cloud and are accessible from any computing device in any location, and mobile wallets run are application installed on mobile computing devices, such as a smart phone. In some embodiments, a hardware wallets store private keys on a hardware device, such as a Universal Serial Bus (USB) storage device. In some embodiments, paper wallets refer to a physical copy or printout of a user's public and private keys. In some embodiments, paper wallets refer to software that is used to securely generate a pair of keys that can be printed.

In some embodiments, digital wallets employ public and private keys for each key address. For example, in some embodiments, the private key is a randomly generated string (numbers and letters), allowing assets to be spent. In some embodiments, the private key is mathematically related to the respective public address, which is a hashed version of the respective public key. In some embodiments, public keys are 256 bits long. In some embodiments, public address for the key (the hash value) is 160 bits long. In some embodiments, the public key is used to ensure only the owner of the address can receive assets. In some embodiments, digital wallets are employed for value transfer, which includes transferring of value by way of, for example, a token or third-party contract call.

1. A utility token system, comprising:
a distributed ledger;
a back-end system; and
a digital wallet associated with a key address, the digital wallet configured to:
provide, to the back-end system, a first transaction comprising a recipient and a token from a plurality of tokens, the token associated with the key address;
the back-end system configured to:
generate a smart contract associated with the tokens, the smart contract comprising a set of instructions which, when invoked with the first transaction and executed, by one or more processors, cause the one or more processors to perform operations comprising:
determining a current supply of the tokens based on a multiplication block stored to the distributed ledger, wherein the token represents a portion of the current supply of the tokens;
determining a valuation of the token based on the current supply of the tokens;
providing the valuation of the token to the recipient;
persist the smart contract to the distributed ledger; and
when receiving the first transaction from the digital wallet, invoking the smart contract with the first transaction.

2. The system of claim 1, wherein the back-end system is configured to:
receive the multiplication block signed by a key associated with a central authority;
verify the signature based on a public address of the key; and
persist the multiplication block to the distributed ledger.

3. The system of claim 2, comprising a peer-to-peer network, the peer-to-peer network comprising a plurality of nodes, wherein the multiplication block is verified by a plurality of the nodes.

4. The system of claim 2, wherein persisting the multiplication block generates a hard fork of the distributed ledger.

5. The system of claim 1, wherein the current supply of the tokens is determined based on an off-chain oracle instead of the multiplication block.

6. The system of claim 1, wherein the back-end system is configure to:
receive, from an affiliate vendor, a second transaction associating the token with the key address;
generate a block comprising the second transaction; and
persist the block to the distributed ledger.

7. The system of claim 6, comprising a peer-to-peer network, the peer-to-peer network comprising a plurality of nodes, wherein the block is verified by a plurality of the nodes.

8. The system of claim 1, wherein the multiplication block comprises a multiplier for the number of tokens.

9. The system of claim 8, wherein the multiplier is determined based on a reference price for each of the tokens.

10. The system of claim 9, wherein the reference price is predetermined.

11. The system of claim 9, wherein the reference price comprises an initial price for the tokens.

12. The system of claim 9; wherein the reference price mirrors a price of an existing currency or cryptocurrency.

13. The system of claim 8, wherein the multiplier comprises a decimal value or a fractional value.

14. The system of claim 1, wherein the recipient comprises an affiliate vendor, and wherein the valuation of the token determines a discount, rebate, or coupon for the affiliate vendor.

15. The system of claim 1, comprising a second digital wallet associated with a second key address, wherein the digital wallet is configured to:
provide a transfer transaction comprising the token and the second key address;
the back-end system configured to:
when receiving the transfer transaction from the digital wallet, generate a transfer block comprising the transfer transaction; and
persist the transfer block to the distributed ledger.

16. The system of claim 15, comprising a peer-to-peer network, the peer-to-peer network comprising a plurality of nodes, wherein the transfer block is verified by a plurality of the nodes.

17. A computer-implemented method of tracking user compliance with a health program, comprising:
storing, in a personal health database at a personal health tracking system, a profile associated with an account belonging to a user, wherein the profile comprises goals related to health;
offering a token to the user, wherein the token represents an encrypted data structure that is processed and stored in a distributed ledger computer network and is redeemable by the user at an affiliate vendor or a health entity in exchange for goods or services provided by the affiliate vendor or health entity;
receiving at least one data unit from the affiliate vendor or health entity comprising information about a transaction between the user and the affiliate vendor or the health entity, the transaction comprising (i) the user earning the token as a reward for healthy behavior or (ii) the user redeeming the token at the affiliate vendor or health entity for the goods or services, where the goods or services assist the user in meeting the goals related to health;
processing the at least one data unit to generate an encrypted data structure and store the encrypted data structure;
generating a hash of the at least one data unit using a cryptographic hash function;
publishing a new transaction comprising the hash of the data to the distributed ledger computing network for validation and addition to the distributed ledger computing network; and
selling the token.

18. The method of claim 17; further comprising:
providing a supply of tokens;
integrating into the distributed ledger computer network an off-chain oracle configured to expand or contract the supply of tokens based on a reference price; and
multiplying the supply of tokens by a number greater than one to expand the supply of tokens if the price of a token exceeds the reference price, or multiplying the supply of tokens by a number less than one to contract the supply of tokens if the price of a token is below the reference price.

19. The method of claim 17; further comprising:
providing a supply of tokens;
providing a special purpose central authority private key configured to expand or contract the supply of tokens; and
multiplying the supply of tokens by a number greater than one to expand the supply of tokens, or multiplying the supply of tokens by a number less than one to contract the supply of tokens.

20. A non-transitory computer readable storage medium comprising computer-executable code configured to cause at least one processor to perform the steps of claim 17.
